(19) United States
(12) Patent Application Publication
(10) Pub. No.: US 2013/0260430 A1
(43) Pub. Date: Oct. 3, 2013

(54) METHOD TO REDUCE GHG EMISSIONS OF FUEL PRODUCTION

(71) Applicant: IOGEN CORPORATION, Ottawa (CA)

(72) Inventor: Patrick J. Foody, Ontario (CA)

(73) Assignee: IOGEN CORPORATION, Ottawa (CA)

(21) Appl. No.: 13/832,618

(22) Filed: Mar. 15, 2013

Related U.S. Application Data
(63) Continuation of application No. 13/688,656, filed on Nov. 29, 2012.
Application No. 13/688,656 is a continuation of application No. 13/688,848, filed on Nov. 29, 2012.
(60) Provisional application No. 61/616,050, filed on Mar. 27, 2012.
(60) Provisional application No. 61/616,060, filed on Mar. 27, 2012.
(60) Provisional application No. 61/715,541, filed on Oct. 18, 2012.
(60) Provisional application No. 61/616,050, filed on Mar. 27, 2012.
(60) Provisional application No. 61/616,060, filed on Mar. 27, 2012.
(60) Provisional application No. 61/715,541, filed on Oct. 18, 2012.

(51) Int. Cl. C12P 7/10

(52) U.S. Cl. CPC C12P 7/10
USPC 435/165

(57) ABSTRACT

The present invention provides a process comprising collecting or sourcing biogenic carbon dioxide from a fermentation that produces a fuel, fuel intermediate or fuel source from organic material. The fermentation may be an anaerobic digestion to produce biogas or a fermentation of sugar to produce a liquid fuel. The biogenic carbon dioxide arising from the fermentation is subsequently supplied to one or more sites that use carbon dioxide in an industrial application for displacement of geologic carbon dioxide which derives a greenhouse gas emissions benefit. Such an industrial application may include using the biogenic carbon dioxide as an additive, a processing agent, a treatment agent, a cooling agent, or a carbon source to make fuels, chemicals or polymers.

### CROSS-REFERENCE TO RELATED APPLICATIONS

This application claims the priority benefit of provisional application No. 61/616,050, filed Mar. 27, 2012, provisional application No. 61/616,060, filed Mar. 27, 2012, provisional application No. 61/715,541, filed Oct. 18, 2012, non-provisional application Ser. No. 13/688,656, filed Nov. 29, 2012 and non-provisional application Ser. No. 13/688,848, filed Nov. 29, 2012, all of which are incorporated herein by reference.

### TECHNICAL FIELD

The present invention relates to a process to reduce the life cycle greenhouse gas emissions associated with a process that transforms organic material into a fuel or a fuel intermediate.

### BACKGROUND OF THE INVENTION

In recent years there has been significant concern about greenhouse gas (“GHG”) emissions and their effect on climate. GHGs, especially carbon dioxide, but also methane and nitrous oxide, trap heat in the atmosphere and thus contribute to climate change. One of the largest sources of GHG emissions is the production and use of fossil fuels for transportation, heating and electricity generation. Another significant source is as a byproduct of certain industrial processes, such as the production of ammonia, or the thermal decomposition of limestone in the manufacture of lime or cement.

Significant efforts have been devoted to reducing the GHG emissions that are associated with production and use of transportation fuels. Renewable fuels, for example, are being used to displace fossil fuels in the transportation sector. Ethanol is the most common renewable fuel, or “biofuel”, currently used for transportation, where it is commonly blended with gasoline at levels from 5% to 85% ethanol. Over 10 billion gallons of ethanol derived from corn were produced in the United States alone in 2010. Another renewable fuel that has been the subject of interest in recent years is biomethane, which is a component of biogas produced by decomposing waste organic material under anaerobic conditions.

Like any other fuel source containing carbon, combustion of renewable fuels such as ethanol releases carbon dioxide into the atmosphere. In addition, the process of fermenting plant derived organic material to produce the fuel will also produce carbon dioxide, which unless captured will enter the atmosphere. These carbon dioxide inputs, however, are considered relatively benign, given that they simply return to the atmosphere carbon that was previously removed therefrom by plant photosynthesis. More generally, this relatively benign nature is also true of any carbon dioxide released due to the combustion, processing or decay of plant matter and other organic material or biological sources, where the underlying carbon had previously been removed from the atmosphere by photosynthesis. Carbon dioxide from such biological sources is generally referred to as “biogenic carbon dioxide.”

Although an unwanted by-product of combustion, carbon dioxide has substantial industrial uses. For example, it is a raw material for the synthesis of various chemicals and polymers, and is used for dry cleaning and as a solvent for organic compounds. There are various non-biogenic commercial sources of carbon dioxide for industrial use. One source is the by-product of other industrial processes, such as the production of ammonia or hydrogen. Another industrial source is from boilers burning fossil fuels. Along with carbon dioxide produced from fossil fuel combustion, carbon dioxide from such industrial processes is referred to as “anthropogenic carbon dioxide.” Unlike biogenic carbon dioxide, release of anthropogenic carbon dioxide into the atmosphere is generally thought to increase concentrations of atmospheric carbon dioxide, life cycle GHG emissions and thereby have an effect on the climate because the underlying carbon is of fossil not atmospheric origin.

A further non-biogenic source of carbon dioxide for industrial use is that which originates from underground reservoirs or deposits. This type of carbon dioxide is produced underground from natural processes. Carbon dioxide from this second source is referred to as “geologic carbon dioxide.” Like anthropogenic carbon dioxide, release of geologic carbon dioxide into the atmosphere is generally thought to increase concentrations of atmospheric carbon dioxide and life cycle GHG emissions, and thereby have an effect on the climate.

A life cycle analysis is often used to determine the overall level of GHG emissions related to a particular fuel. Such life cycle analyses seek to account for the GHG fluxes associated with each stage of the development, production, delivery and use of the fuel. Biofuels are derived from organic material that contains carbon removed from the atmosphere during photosynthesis. In life cycle analyses, the carbon dioxide removed from the atmosphere during photosynthesis is credited against the carbon dioxide released during combustion, leading to lower net levels of GHG emissions. By contrast, fossil fuels such as petroleum or coal are extracted from beneath the earth, and, when they are burned, release carbon into the atmosphere, which adds to total atmospheric GHG.

The United States government, through the Energy Independence and Security Act (“EISA”) of 2007, has promoted the use of renewable fuels with reduced GHG emissions. Some of the purposes of the act are to increase the production of clean renewable fuels, to promote research on and deploy GHG capture and to reduce fossil fuels present in transportation fuels. The act sets out a Renewable Fuels Standard (“RFS”) with increasing annual targets for the renewable content of transportation fuel sold or introduced into commerce in the United States. The RFS mandated volumes are set by four nested fuel category groups, namely renewable biofuel, advanced biofuel, biomass-based diesel, and cellulosic biofuel, which require at least 20%, 50%, 50% and 60% GHG reductions relative to gasoline, respectively. The United States Environmental Protection Agency (“EPA”) conducts life cycle analyses to determine whether or not renewable fuels produced under varying conditions will meet these GHG thresholds.

The mandated annual targets of renewable content in transportation fuel under the RFS are implemented using a credit called a Renewable Identification Number, referred to herein as a “RIN”, to track and manage the production, distribution and use of renewable fuels for transportation purposes. RINs can be likened to a currency used by obligated parties to certify compliance with mandated renewable fuel volumes. The EPA is responsible for overseeing and enforcing blending mandates and developing regulations for the generation, trading and retirement of RINs.

In addition to EISA, numerous jurisdictions, such as the state of California, the province of British Columbia, Canada and the European Union, have set annual targets for reduction in average life cycle GHG emissions of transportation fuel. Such an approach is often referred to as a Low Carbon Fuel Standard (“LCFS”), where credits may be generated for the use of fuels that have lower life cycle GHG emissions than a specific baseline fuel. Such fuels are often referred to as having a lower “carbon intensity” or “CI”.

The use of carbon dioxide in industry can result in a portion of the gas being sequestered and a portion being released to the atmosphere. Such release of carbon dioxide may result from known or intentional release, such as when carbonated drinks are opened, or release can occur as a result of leakages such as “fugitive emissions”, which originate from equipment leakages, and other unintended or irregular release of carbon dioxide depending on the particular industrial application. Regardless of the particular application, when using carbon dioxide in industry, there are often uncertainties regarding what fraction of the carbon dioxide is captured and prevented from release to the atmosphere and what fraction is released. These uncertainties can limit the acceptance of the use of carbon dioxide in industry as a means of reducing the measured life cycle GHG emissions. Given the undisputable concern with carbon dioxide's deleterious effects on climate, but given the indisputable industrial role for carbon dioxide in modern society, there is a pressing need to satisfy that role in a more environmentally benign manner. There is a need in the art for a cost-effective technology to enable biofuel producers to reduce GHG emissions and preferably contribute to reducing GHG emissions to levels that are at least about 50% lower than a “gasoline baseline”, which is a value representing the life cycle GHG emissions for gasoline set by government authorities. There is a further need to enable producers of a fuel, or an intermediate thereof, produced by fermentation to qualify for desired credits associated with reduced GHG life cycle emissions, including for RINs under EISA having higher market value and associated with lower GHG emissions and LCFS credits.

### SUMMARY OF THE INVENTION

The present invention seeks to overcome or ameloriate the shortcomings of known processes for reducing the GHG emissions associated with a fuel or fuel intermediate.

According to the invention, biogenic carbon dioxide is collected from a fermentation that produces a fuel or fuel intermediate from organic material. The fermentation may be an anaerobic digestion to produce biogas or a fermentation of sugar to produce a liquid fuel or fuel intermediate. Regardless of the nature of the fermentation, each of these processes generates biogenic carbon dioxide. The biogenic carbon dioxide arising from the fermentation is subsequently supplied to one or more sites that use carbon dioxide in an industrial application for displacement of geologic carbon dioxide. Such an industrial application may include using the biogenic carbon dioxide as an additive, a processing agent, a treatment agent, a cooling agent, or a carbon source to make fuels, chemicals or polymers.

The displacement of geologic carbon dioxide with biogenic carbon dioxide provides for significant reductions in GHG emissions of the fuel or fuel intermediate.

As discussed, when carbon dioxide is used in industry, a certain amount of this gas is released into the atmosphere, and a certain amount is captured and prevented from such release. Because geologic carbon dioxide originates from underground reservoirs or deposits, when this type of carbon dioxide leaks, or is otherwise emitted to the atmosphere, the resultant emissions need to be accounted for in life cycle GHG calculations. However, as mentioned, it can be difficult to quantify with precision the fraction of carbon dioxide released to the atmosphere and the fraction which is captured and removed from the atmosphere. This can in turn lead to uncertainties when calculating life cycle GHG emissions.

By displacing geologic carbon dioxide with biogenic carbon dioxide in accordance with the invention, life cycle GHG emission calculations need not determine the proportion of the carbon dioxide that is released and that which is captured and more permanently removed from the atmosphere; in all cases, the credits and debits of typical GHG accounting lead to a GHG saving equal to the amount of biogenic carbon dioxide collected and used in the industrial application. That is, the savings occur independently of the mix of released carbon dioxide and that which is removed from the atmosphere.

By contrast, when biogenic carbon dioxide is used in an industrial application without displacement of geologic carbon dioxide, the savings are lower and must account for any leakage. To illustrate, an example of a life cycle GHG emission analysis in which biogenic carbon dioxide is used in an industrial application without displacement of geologic carbon dioxide is set out below.

In this example, if for a given amount of carbon dioxide introduced into a site that uses carbon dioxide in an industrial application (typically measured as a flow rate), the percentage of carbon dioxide ultimately leaked to the atmosphere is X %, then the remainder of the introduced amount (100%−X %) is not leaked. A life cycle analysis of the carbon dioxide emissions related to the use of 100 units of biogenic carbon dioxide in an industrial application, without implementing the invention may include:

(a) a credit for the amount of biogenic carbon dioxide collected and used in the industrial application (100 units); and
(b) a debit for emissions related to biogenic carbon dioxide that is leaked (X units, given leakage is X % of the input flow).

In the above case, the net GHG impact is an improvement of only 100−X.

By contrast, the net GHG impact by practicing the invention is 100, i.e., accounting for X is not required. To illustrate, a life cycle analysis of the method of the invention involving the displacement of 100 units of geologic carbon dioxide with 100 units of biogenic carbon dioxide comprised of calculating the emissions impact of the disposition of the biogenic carbon dioxide and crediting the emissions impact of the displaced geologic carbon dioxide, may include:

(a) a credit for the amount of biogenic carbon dioxide collected and used in the industrial application (100 units);
(b) a debit for the amount of biogenic carbon dioxide that is leaked from the site (X units, given leakage is X % of the input flow); and
(c) a credit for the emissions impact of the avoided amount of geologic carbon dioxide released to the atmosphere, equal to X units, comprised of the following:

(i) emissions related to geologic carbon dioxide that would have been leaked from the use of the same amount of geologic carbon dioxide (X units, given leakage is X % of the input flow); and

(ii) zero net emissions for geologic carbon dioxide that remains sequestered from the atmosphere from the use of the same amount of geologic carbon dioxide, because such geologic carbon dioxide would have been originally extracted from underground, but then remains sequestered from the atmosphere when used in the industrial application, thus providing no net emissions impact.

The net GHG benefit when implementing the invention would be calculated as the credit in (a) (100 units) minus the debit in (b) (X units) plus the credit in (c) (X units), i.e., 100−X+X=100. The debit in (b) is offset by the credit in (c), and thus the overall net reduction in emissions (100 units, in this example) is independent of the amount of carbon dioxide that is leaked or otherwise emitted in the system.

For example, in an ethanol fermentation process where 36.12 grams of carbon dioxide is generated per Mega Joule of fuel (g CO₂ eq/MJ), when 50 wt % of the amount of carbon dioxide evolved during fermentation is collected and used in accordance with the invention, the savings in GHG emissions can be as high as 18.06 g CO₂ eq/MJ relative to a production process baseline, assuming there are no carbon dioxide losses associated with collection, compression, transportation or other processing. Thus, the overall net reduction in emissions is equal to the amount of biogenic carbon dioxide used to displace geologic carbon dioxide, without any deductions for or quantification of carbon dioxide release or leakage.

As used herein, a “production process baseline” refers to the life cycle carbon dioxide emissions associated with a corresponding “fermentation based fuel production process” conducted under identical conditions except the biogenic carbon dioxide that is evolved is released to the atmosphere. By a “fermentation based fuel production process”, or “fuel fermentation”, it is meant a fermentation in which organic material or a substance derived from organic material, for example sugar, is fermented to produce a fuel or fuel intermediate and biogenic carbon dioxide. A “fermentation based fuel” as used herein is a fuel or fuel intermediate produced by such fermentation.

Reductions in life cycle GHG emissions are not achieved when the biogenic carbon dioxide is being used to displace anthropogenic carbon dioxide. This is because displacing anthropogenic carbon dioxide cannot yield any credit associated with avoiding release of carbon dioxide since the release of carbon dioxide into the atmosphere is not avoided. That is, the anthropogenic carbon dioxide will be released to the atmosphere regardless of whether it is used in the industrial application, while avoiding geologic carbon dioxide use in industry means that it remains underground.

The invention is not bound to any one particular method for calculating life cycle GHG emissions. In life cycle analyses, the energy consumed and emissions generated by a fuel production process, for example, an ethanol plant, would be allocated not only to the fuel, but also to each of the by-products and there are a number of methods that can be used to estimate by-product allocations. These include methods that account for the energy usage of each by-product, based on engineering analysis of the processes related to each by-product. As would be understood by those skilled in the art, the life cycle net carbon emissions associated with a fuel or fuel intermediate would be calculated and data generated in accordance with the prevailing applicable guidelines which may vary by regulatory standard or change over time. The guidelines for such calculations would be known to those skilled in the art.

The process of the invention not only has positive environmental implications, but also allows fuel production facilities, or other parties, to qualify for more desirable renewable fuel credits than could otherwise be attained. This includes the generation of RINs under EISA having higher market value, such as RINS having a D code of 3 or 5, or greater amounts of renewable fuel credits under the LCFS.

Thus, according to a first aspect of the invention, there is provided a process for reducing life cycle GHG emissions associated with production of a liquid fuel or fuel intermediate comprising: (i) producing sugar from plant derived organic material; (ii) fermenting the sugar to produce biogenic carbon dioxide and the liquid fuel or fuel intermediate; (iii) collecting an amount of biogenic carbon dioxide generated from the step of fermenting; (iv) supplying the biogenic carbon dioxide from step (iii) to one or more sites that use carbon dioxide in an industrial application, and causing displacement of geologic carbon dioxide; (v) recovering the liquid fuel or fuel intermediate produced by the step of fermenting; and (vi) generating a renewable fuel credit associated with the liquid fuel or fuel intermediate; (vii) prior to step (vi), generating or receiving data representative of a life cycle GHG emission reduction of the liquid fuel or fuel intermediate relative to a gasoline baseline, wherein the life cycle GHG emissions associated with the production of the liquid fuel or fuel intermediate are reduced by at least 1.5 g CO₂ eq/MJ relative to a production process baseline as a result of the displacement.

According to embodiments of this aspect of the invention, the life cycle GHG emissions associated with the production of the liquid fuel or fuel intermediate are reduced by between about 2 g CO₂ eq/MJ and about 35 g CO₂ eq/MJ relative to a production process baseline as a result of the displacement of geologic carbon dioxide.

According to a second aspect of the invention, there is provided a process to reduce the life cycle GHG emissions associated with production of a liquid fuel or fuel intermediate, the process comprising: (i) producing sugar from plant derived organic material and converting the sugar to the liquid fuel or fuel intermediate in a fuel production facility; (ii) using methane to supply energy in any part of the fuel production facility or associated utilities, wherein the methane has associated with it life cycle GHG emissions that are reduced relative to a biomethane production process baseline as a result of the practice, or arrangement of the practice by one or more third parties, of: (a) anaerobically digesting plant derived organic material to produce biogas comprising biomethane and biogenic carbon dioxide; (b) separating the biomethane and biogenic carbon dioxide; (c) collecting an amount of the biogenic carbon dioxide generated from the step of separating; and (d) supplying the biogenic carbon dioxide from step (c) to one or more sites that use carbon dioxide in an industrial application, and causing displacement of geologic carbon dioxide; and (e) supplying the biomethane to an apparatus for delivering methane to one or more fuel production facilities; (iii) recovering the liquid fuel or fuel intermediate; and (iv) generating a renewable fuel credit associated with the liquid fuel or fuel intermediate; (v) prior to step (iv) generating or receiving data representative of a life cycle GHG emission reduction of the liquid fuel or fuel intermediate relative to a gasoline baseline, wherein the life cycle GHG emissions associated with the production of the biomethane are reduced by at least 5 g CO₂ eq/MJ relative to a biomethane production process baseline as a result of the displacement of geologic carbon dioxide.

The “biomethane production process baseline” refers to the life cycle GHG emissions associated with a biogas production process conducted under identical conditions except the biogenic carbon dioxide that is separated from the biomethane is released to the atmosphere.

Steps (a)-(e), step (v) or steps (a)-(e) and (v) may be practiced by one or more third parties.

The methane used to supply energy in any part of the fuel production facility or associated utilities may be withdrawn from a natural gas pipeline containing methane from sources other than anaerobic digestion of organic material. The methane may be used to supply energy in the form of heat or electricity.

According to a further embodiment of the second aspect of the invention, biogenic carbon dioxide resulting from converting the sugar to the liquid fuel or fuel intermediate in step (i) is collected for use in one or more sites that use carbon dioxide in an industrial application for displacement of geologic carbon dioxide.

The life cycle GHG emissions associated with the production of the biomethane may be reduced by between about 5 g CO₂ eq/MJ and about 35 g CO₂ eq/MJ relative to a production process baseline as a result of the displacement of geologic carbon dioxide.

According to embodiments any of the foregoing aspects of the invention, the plant derived organic material for producing sugar is starch. In certain embodiments of the invention, the plant derived organic material for producing sugar is derived from wheat, barley, rye, sorghum, rice, potato, sugar beet or sugar cane. In further embodiments, if the liquid fuel or fuel intermediate is ethanol, the sugar is produced from organic material that is non-corn starch or predominantly non-corn starch. Preferably, the organic material that is non-corn starch is wheat or sorghum. In another embodiment of the invention, the liquid fuel or fuel intermediate is butanol or isobutanol from corn starch.

According to a further embodiment of either aspect of the invention, the liquid fuel or fuel intermediate is an alcohol. The alcohol may be ethanol, propanol, butanol, or isobutanol.

According to embodiments of any of the foregoing aspects of the invention, the one or more sites use carbon dioxide as an additive, a processing agent, a treatment agent, a cooling agent, or a carbon source to make fuels, chemicals or polymers. The carbon dioxide may be used as an additive to a food, a beverage or water, as a processing agent to process a food or food ingredient, as a carbon source to make a carbonate or methanol, or as a cooling agent in food processing or preservation. According to one embodiment of the invention, the biogenic carbon dioxide is compressed and purified after its collection.

According to a further embodiment of any of the above aspects of the invention, the displacement results from taking out of use a first amount of geologic carbon dioxide at the one or more sites that use carbon dioxide in an industrial application and subsequently using the biogenic carbon dioxide that is supplied to displace the first amount of geologic carbon dioxide.

According to further embodiments, the displacement results from: (a) introducing the biogenic carbon dioxide into an apparatus for transporting carbon dioxide to one or more sites that used or are using geologic carbon dioxide in an industrial application; or (b) supplying the biogenic carbon dioxide for use in one or more sites that used or are using geologic carbon dioxide in an industrial application.

According to another embodiment of either aspect of the invention, the step of supplying comprises introducing the biogenic carbon dioxide into apparatus for transporting the biogenic carbon dioxide to the one or more sites, wherein in respect of at least one or more of the sites at least two conditions are met selected from: (a) the site has used geologic carbon dioxide in the industrial application; (b) the site has access to geologic carbon dioxide for use in the industrial application; and (c) written documentation indicates that biogenic carbon dioxide is used to displace geologic carbon dioxide. Preferably, in respect of at least one or more of the sites, written documentation indicates that biogenic carbon dioxide is used to displace geologic carbon dioxide. The written documentation may be in electronic format.

The data representative of a life cycle GHG emission reduction of the liquid fuel or fuel intermediate may be stored in computer readable format in a storage medium used to retain digital data, such as a drive in a computer or a disk. Such data may be characterized in that it does not take into account emissions due to any release of the carbon dioxide to the atmosphere during or after its use in an industrial application.

According to yet further embodiments of the invention, the data representative of a life cycle GHG emission reduction of the liquid fuel or fuel intermediate relative to a gasoline baseline is determined by a quantification of a GHG emission reduction due to a reduction in the use of geologic carbon dioxide in the one or more sites that occurred or would occur over the lifetime of the one or more sites as a result of the use of biogenic carbon dioxide.

Preferably, the life cycle GHG emissions associated with the fuel or fuel intermediate are less than 50% measured relative to a gasoline baseline.

The renewable fuel credit generated in either aspect of the invention may be a renewable identification number. The renewable identification number may have a D code value of 3 or 5. According to one embodiment of the invention, the renewable identification number is not separated from the fuel or fuel intermediate. In another embodiment of the invention, the renewable fuel credit is a low carbon fuel credit.

The present invention also provides a process for generating a D5 RIN credit associated with ethanol produced in an ethanol production facility, the process comprising using a non-corn starch feedstock, or a predominantly non-corn starch feedstock to supply the production facility, and carrying out the process of either of the above aspects of the invention to reduce the life cycle GHG emissions of the ethanol to a level relative to a gasoline baseline sufficient to qualify for the D5 RIN credit.

According to another aspect of the invention, there is provided a process comprising: (i) receiving carbon dioxide for use at a site that uses carbon dioxide in an industrial application, the carbon dioxide produced by the process of any of the foregoing aspects or embodiments of the invention; and (ii) using the carbon dioxide received in step (i) to displace geologic carbon dioxide. Preferably, the carbon dioxide received for use at the site is produced by a third party.

According to yet another aspect of the invention, there is provided a process comprising: (a) withdrawing an amount of carbon dioxide from an apparatus for delivering carbon dioxide to one or more sites that use carbon dioxide in an industrial application, the apparatus having had introduced thereto an amount of biogenic carbon dioxide derived from a fermentation that produces a liquid fuel or fuel intermediate using organic material as a feedstock, the carbon dioxide withdrawn having GHG emission attributes associated therewith that are the same as the GHG emission attributes of the biogenic carbon dioxide introduced to the apparatus; and (b) using the carbon dioxide withdrawn in step (a) to displace geologic carbon dioxide. Preferably, the carbon dioxide received for use at the site is produced by a third party.

According to a further aspect of the invention, there is provided a process comprising: (a) withdrawing an amount of carbon dioxide from an apparatus for delivering carbon dioxide to one or more sites that use carbon dioxide in an industrial application, the apparatus having had introduced thereto an amount of biogenic carbon dioxide derived from an anaerobic digestion of organic material, the carbon dioxide withdrawn having GHG emission attributes associated therewith that are the same as the GHG emission attributes of the biogenic carbon dioxide introduced to the apparatus; and (b) using the carbon dioxide withdrawn in step (a) at a site that uses carbon dioxide in the industrial application to displace geologic carbon dioxide.

By “the carbon dioxide withdrawn having GHG emission attributes associated therewith that are the same as the GHG emission attributes of the biogenic carbon dioxide introduced to the apparatus”, it is meant that although the carbon dioxide withdrawn from the apparatus may not contain actual molecules from the original organic material from which the biogenic carbon dioxide is derived, it is still considered to have at least substantially the same GHG emissions as the biogenic carbon dioxide introduced to the apparatus. For example, the withdrawal of non-biogenic carbon dioxide from a pipeline that is fed by both biogenic carbon dioxide and non-biogenic sources of carbon dioxide may be considered by regulators to possess the same GHG attributes of the biogenic carbon dioxide fed to the pipeline. As discussed further herein, for such GHG emission attributes to be recognized, the amount of carbon dioxide introduced to the apparatus and the amount withdrawn are preferably the same and may be evidenced by written documentation.

In an embodiment of the invention, the amount of carbon dioxide withdrawn is less than or at least “approximately equal” to the amount of biogenic carbon dioxide introduced to the apparatus. By “approximately equal” it is meant that the amount of carbon dioxide withdrawn does not vary by more than 10%, more preferably by more than 5% by weight of the amount of carbon dioxide introduced to the apparatus.

According to certain embodiments of this aspect of the invention, the displacement results from taking out of use a first amount of geologic carbon dioxide at the site that uses carbon dioxide in an industrial application and subsequently using the carbon dioxide that is withdrawn to displace the first amount of geologic carbon dioxide. According to further embodiments, the biogenic carbon dioxide is sourced from a fuel production facility that generates renewable fuel credits associated with producing a liquid fuel or fuel intermediate.

The displacement of step (b) may result from taking out of use a first amount of geologic carbon dioxide at the site that uses carbon dioxide in an industrial application and subsequently using the carbon dioxide that is withdrawn to displace the first amount of geologic carbon dioxide.

According to another aspect of the invention, there is provided a process comprising: (i) receiving carbon dioxide for use at a site that uses carbon dioxide in an industrial application, said carbon dioxide supplied from an anaerobic digestion that produces biogas comprising biomethane and biogenic carbon dioxide; and (ii) using the carbon dioxide received in step (i) to displace geologic carbon dioxide.

According to another aspect of the invention, there is provided a process for reducing life cycle GHG emissions associated with production of a liquid fuel or fuel intermediate comprising: (i) producing sugar from plant derived organic material; (ii) fermenting the sugar to produce biogenic carbon dioxide and the liquid fuel or fuel intermediate; (iii) collecting an amount of biogenic carbon dioxide generated from the step of fermenting; (iv) supplying the biogenic carbon dioxide from step (iii) to one or more sites that use carbon dioxide in an industrial application, and causing displacement of geologic carbon dioxide; wherein the life cycle GHG emissions associated with the production of the liquid fuel or fuel intermediate are reduced by at least 1.5 g CO₂ eq/MJ relative to a production process baseline as a result of the displacement; (v) recovering the liquid fuel or fuel intermediate produced by the step of fermenting; (vi) generating or receiving data relating to a quantity of carbon dioxide displaced or a life cycle GHG emission analysis of the liquid fuel or fuel intermediate resulting from the fermentation; and (vii) generating a renewable fuel credit associated with the liquid fuel or fuel intermediate.

According to a further aspect of the invention, there is provided a process to reduce the life cycle GHG emissions associated with production of a liquid fuel or fuel intermediate, the process comprising: (i) producing sugar from plant derived organic material and converting the sugar to the liquid fuel or fuel intermediate in a fuel production facility; (ii) using methane to supply energy in any part of the fuel production facility or associated utilities, wherein the methane has associated with it life cycle GHG emissions that are reduced relative to a biomethane production process baseline as a result of the practice of: (a) anaerobically digesting plant derived organic material to produce biogas comprising biomethane and biogenic carbon dioxide; (b) separating the biomethane and biogenic carbon dioxide; (c) collecting an amount of the biogenic carbon dioxide generated from the step of separating; and (d) supplying the biogenic carbon dioxide from step (c) to one or more sites that use carbon dioxide in an industrial application, and causing displacement of geologic carbon dioxide; and (e) supplying the biomethane to an apparatus for delivering methane to one or more fuel production facilities; wherein the life cycle GHG emissions associated with the production of the biomethane are reduced by at least 5 g CO₂ eq/MJ relative to a biomethane production process baseline as a result of the displacement of geologic carbon dioxide; (iii) recovering the liquid fuel or fuel intermediate; (iv) generating data or receiving data relating to a quantity of carbon dioxide displaced or a life cycle GHG emission analysis of the liquid fuel or fuel intermediate; and (v) generating a renewable fuel credit associated with the liquid fuel or fuel intermediate.

According to any of the foregoing aspects of the invention, the GHG emission reductions are due to displacement of the extraction of geologic carbon dioxide or use of geologic carbon dioxide at the site.

The present invention also provides methods for generating or receiving data relating to a life cycle GHG emission analysis of a fuel or fuel intermediate having reductions in life cycle GHG emissions due to the practice of the invention. As used herein, the term “data” refers to information in numerical format. The data may be stored in digital format in a storage medium used to retain digital data.

As used herein, “data relating to”, with reference to a GHG emission analysis includes any data that would be inputted to a life cycle GHG emission analysis of a fuel or fuel intermediate or any data that uses values reliant upon or calculated from the life cycle GHG emission analysis. Examples of data that is inputted to a life cycle GHG emission analysis includes the following: GHG emissions in production and recovery of the fuel or fuel intermediate, energy use associated with feedstock transportation, emissions from fuel or fuel intermediate production, transport and storage of the fuel or fuel intermediate prior to its use in transportation or for heating, and the like. Examples of data that use values reliant upon or calculated from a life cycle GHG emission analysis include one or more of the following: the weight amount (e.g., in tonnes) of carbon dioxide emissions reduced by the practice of the invention; the volumes (e.g., in gallons) of fuel or fuel intermediate produced or generated using the method of the invention to reduce GHG emissions; the aggregate number or rate of credits generated as a result of using the method of the invention to reduce GHG emissions; and data describing the eligibility of the method of the invention for credits such as database fields identifying the method through a numerical value.

Data relating to a quantity of carbon dioxide displaced may be a numerical value representing a quantity of geologic carbon dioxide displaced in an industrial application, such as a numerical value in g CO₂ eq/MMBTU or CO₂ eq/MJ of fuel produced.

According to a further aspect of the invention, there is provided a process comprising: (i) providing an amount of biogenic carbon dioxide generated from a fermentation process to produce a liquid fuel or fuel intermediate; (ii) supplying the biogenic carbon dioxide from step (i) to one or more sites that use carbon dioxide in an industrial application; (iii) generating data or receiving data in written documentation from a third party, said data being representative of a life cycle GHG emission reduction of the liquid fuel or fuel intermediate resulting from the fermentation relative to a gasoline baseline, wherein the data demonstrates a reduction in emissions due to displacement of geologic carbon dioxide, the data is stored in digital format in a storage medium used to retain digital data, and the life cycle GHG emissions associated with the production of the liquid fuel or fuel intermediate are reduced by at least 1.5 g CO₂ eq/MJ relative to a production process baseline as a result of the displacement, (iv) recovering the liquid fuel or fuel intermediate produced by the fermentation process; and (v) generating a renewable fuel credit associated with the liquid fuel or fuel intermediate.

### BRIEF DESCRIPTION OF THE FIGURES

FIG. 1 is a comparison of the life cycle GHG emissions for a gasoline baseline and ethanol produced from a fermentation of sugar, where the sugar is derived from grain sorghum. Bar A is the gasoline baseline; bar B is a production process baseline in which ethanol is produced from the fermentation of sugar from grain sorghum and in which the biogenic carbon dioxide from the fermentation is not collected; and bar C is a process in which biogenic carbon dioxide is collected from the fermentation and used to displace geologic carbon dioxide in an industrial application in accordance with embodiments of the invention.

FIG. 2 is a comparison of the life cycle GHG emissions for a gasoline baseline and ethanol produced from a fermentation of sugar, where such sugar is derived from grain sorghum. Bar A is the gasoline baseline; bar B is a production process baseline for the ethanol; bar C is ethanol produced from a process in which methane used for energy in the ethanol production process originates from an anaerobic digestion which produces biomethane and biogenic carbon dioxide and in which carbon dioxide is not collected (biomethane production process baseline); and bar D is ethanol from a process in which the methane used in the production process originates from an anaerobic digestion which produces biomethane and biogenic carbon dioxide and in which biogenic carbon dioxide is collected and used to displace geologic carbon dioxide in an industrial application in accordance with embodiments of the invention.

### DETAILED DESCRIPTION OF THE INVENTION

The following description is of a preferred embodiment by way of example only and without limitation to the combination of features necessary for carrying the invention into effect. The headings provided are not meant to be limiting of the various embodiments of the invention.

### Organic Material

The first step in the present invention is to produce biogenic carbon dioxide. Any suitable biologic source material derived from plants or animals can be used as the organic material in the process of the invention to provide a carbon and/or energy source for the fermentation to produce biogenic carbon dioxide. This includes plant derived organic material comprising polysaccharides, including starch, cellulose and hemicellulose, oligosaccharides, disaccharides, monosaccharides, or a combination thereof. Other biologic source material that can be utilized as a carbon and/or energy source for the fermentation includes compounds or molecules derived from organic material, such as lignin and fats.

According to a preferred embodiment of the invention, the plant derived organic material includes material comprising starches, sugars or other carbohydrates, including sugar and starch crops. The sugar and starch crops may include, but are not limited to, corn, wheat, barley, rye, sorghum, rice, potato, cassava, sugar beet, sugar cane, or a combination thereof. In a preferred embodiment, the sugar or starch crop is non-corn starch or predominantly non-corn starch meaning no greater than 20 wt % of the organic material comprises starch from corn kernels. According to some embodiments, if the fuel is ethanol, the organic material is not corn starch. According to such an embodiment, the plant derived organic material includes wheat, barley, rye, sorghum, rice, potato, cassava, sugar beet, sugar cane or a combination thereof. Preferably, the plant derived organic material is wheat or sorghum. In another embodiment of the invention, the ethanol is derived from wheat or sorghum.

According to some embodiments of the invention, the organic material originates from a waste stream such as landfill material, including food and yard waste that may or may not be intermixed with non-organic components of landfill material; agricultural waste, including animal waste material such as manure; slaughterhouse waste and fish waste, waste from plant operations, including sewage sludge, still bottoms or other waste streams from fermentation plants; or a combination thereof.

It is possible, but less preferred, to use lignocellulosic feedstock as the organic material, such as agricultural residues for example, soybean stover, corn stover, rice straw, sugar cane straw, rice hulls, barley straw, corn cobs, wheat straw, canola straw, oat straw, oat hulls, corn fiber or a combination or derivative thereof; cultivated crops, for example, grasses such as C4 grasses; sugar processing residues, for example, bagasse, such as sugar cane bagasse, beet pulp or a combination or derivative thereof; and woody plant biomass such as forestry products.

### Preparation of the Organic Material for Fermentation

Prior to fermentation, the organic material may be processed by mechanical, chemical, thermal and/or biological processes. Fermentable material may be obtained from source material using techniques that are known to those of ordinary skill in the art, including, but not limited to those described below.

In some embodiments of the invention, plant derived organic material is processed to produce sugar. The sugar in turn is fermented to produce the fuel or fuel intermediate. Sugar crops, including, but not limited to, sugar cane, sugar beets or sweet sorghum, may be subjected to a mechanical treatment, such as crushing and/or pressing, to extract the sugar from the plants. For example, sucrose from sugar cane can be extracted using roller mills. Sugar from sweet sorghum stalks can be extracted in a similar manner, although certain varieties of sorghum contain grain that can be processed using technology employed for processing starch crops as described below.

Starch crops, which include cereal crops, may be subjected to size reduction, such as by milling or grinding. The starch may be subsequently hydrolyzed with enzymes, by chemical treatment, or some combination of these treatments. By way of example, grain may be milled with a roller or hammer mill, followed by the addition of liquid and hydrolysis of the starch with amylase to produce fermentable sugar. This method is commonly referred to as “dry milling”. An alternative method is wet milling in which the grain is steeped, such as in an acidic solution and/or a solution containing enzymes, and then subjected to size reduction, such as milling, to facilitate separation of the starch from the other components of the grain. The starch is subsequently hydrolyzed to sugar using methods described above.

The production of fermentable sugar from lignocellulosic feedstocks can be carried out by any of a variety of techniques known to those of skill in the art. For example, pretreatment followed by hydrolysis involving enzymatic or chemical treatment including by acid or alkali treatment, can be utilized.

Those of ordinary skill understand that the embodiments and examples discussed herein are non-limiting, and accordingly that other known or later-developed technologies for processing the plant derived organic source material to produce sugar, may be utilized in conformity with the present invention.

### Fermentation

Fermentation of the organic material yields a fermentation based fuel and biogenic carbon dioxide. The fermentation based fuel includes any product or byproduct of the fermentation used as a fuel or as a fuel intermediate. A fuel intermediate is a precursor used to produce a fuel by a further conversion process, such as by fermentation or chemical reaction. The fermentation based fuel may be a liquid fuel or fuel intermediate, such as an alcohol, or a gaseous fuel or fuel intermediate produced by fermentation, such as biomethane.

Non-limiting examples of liquid fuels or fuel intermediates that can be used in accordance with the invention include alcohols such as ethanol, propanol, butanol and isobutanol. Most preferably, the alcohol is ethanol. Also preferred is ethanol that is not made from corn starch, but other plant derived organic material. The gaseous fuel or fuel intermediate may be produced by anaerobic digestion, as set out below. Hydrogen may also be produced from organic material in accordance with the invention. The fuel includes, but is not limited to, transportation fuel or heating fuel. The fuel may be for use in motor vehicles, motor vehicle engines, non-road vehicles or non-road engines, jets and for heating applications.

The fermentation utilized to generate the biogenic carbon dioxide of the present invention can be conducted using any suitable biocatalyst, including fermentation microorganisms selected from yeast, fungi and bacteria. The organic material that serves as the carbon and/or energy source for the fermentation may be plant derived or derived from animals, such as animal waste products, as set forth above.

The fermentation may be conducted in batch, continuous or fed-batch modes with or without agitation. Preferably, the fermentation reactors are agitated lightly with mechanical agitation. A typical commercial-scale fermentation may be conducted using multiple reactors. The fermentation microorganisms may be recycled back to the fermentor or may be sent to downstream processes without recycle.

Although the process conditions can vary, in one embodiment of the process of the present invention, the fermentation is performed at or near the temperature and pH optimum of the fermentation microorganism. Without being limiting, a typical temperature range for yeast fermenting glucose is between about 25° C. and about 35° C.; however, the temperature may be higher if the yeast is naturally or genetically modified to be thermostable. For anaerobic digestion, a typical temperature range is often higher, such as between about 50° C. and about 70° C. The amount of the fermentation microorganism used to inoculate the fermentation may depend on factors such as the activity of the fermentation microorganism, the desired fermentation time, the volume of the reactor and other parameters. It will be appreciated that these parameters may be adjusted to achieve the desired fermentation conditions.

The fermentation may also be supplemented with additional nutrients required for the growth of the fermentation microorganism. For example, yeast extract, specific amino acids, phosphate, nitrogen sources, salts, trace elements and vitamins may be added to the fermentation to support their growth.

The fermentation organism or biocatalyst used may depend on the substrate and the fermentation based fuel that is produced. For ethanol production, the fermentation may be carried out with any microorganism suitable for such purpose, including yeast and bacteria. Saccharomyces spp. yeast is a typical biocatalyst for ethanol production, although other biocatalysts may be used to produce the fermentation based fuel. Ethanol production can also be carried out with bacteria such as Escherichia coli, Klebsiella oxytoca and Zymomonas mobilis. Butanol may be produced from glucose by a microorganism such as a bacterium, including Escherichia coli or Clostridium acetobutylicum. Propanol production can be carried out using bacteria, such as Escherichia coli. Isobutanol can be produced fermentatively by yeast, including those described in WO 2010/075504.

The product of the fermentation is preferably used as a fuel itself. Alternatively, the product of the fermentation can be utilized as a fuel intermediate. For example, processes are known for converting isobutanol produced fermentatively to fuels, including hydrocarbon fuels such as jet fuel, diesel and gasoline.

The fermentation may be an anaerobic digestion, which is the biologic breakdown of organic material by microorganisms under low oxygen conditions, or in the absence of oxygen, to produce gases. The gases produced by anaerobic digestion of organic material include biogenic carbon dioxide and “biogas” comprising biomethane, also referred to herein as biogas derived methane or renewable natural gas. Other gases may be generated during anaerobic digestion as well, such as hydrogen. As would be appreciated by those skilled in the art, anaerobic digestion generally involves the decomposition of waste organic material, including carbohydrates, fats and proteins therein, into simple sugars and glycerol. These compounds are then converted to acids, which are subsequently converted into biomethane by methanogenic bacteria or other microorganisms. Biomethane can be used as a fuel itself or used to produce other fuels, as described in co-pending U.S. non-provisional application Ser. No. 13/721,157, which is incorporated herein by reference in its entirety.

The biogas may be produced at a municipal or industrial operation. This includes, without limitation, a landfill, a waste treatment facility, such as a sewage treatment facility, and a manure digestion facility, such as a facility located on a farm or a facility that processes materials collected from farms. The digestion may or may not be contained within an anaerobic digester.

The biogas and biogenic carbon dioxide is optionally derived from landfill waste. Landfill biogas may be produced by organic material decomposing under anaerobic conditions in a landfill. The waste is covered and mechanically compressed by the weight of the material that is deposited from above. This material prevents oxygen exposure thus allowing anaerobic microbes to thrive. By appropriately engineering a collection system at the landfill site, the resultant biogas and biogenic carbon dioxide is captured. Biogas and biogenic carbon dioxide can also be produced from organic material that is separated from waste that otherwise goes to landfills. According to further embodiments of the invention, the biogas production site contains an anaerobic digester for digesting the waste.

### Collection of Biogenic Carbon Dioxide

In accordance with the present invention, after production the biogenic carbon dioxide is collected for later use. Collection of biogenic carbon dioxide from a fuel fermentation can be conducted in any manner sufficient to ensure that a desired level of carbon dioxide evolved or generated from the fermentation is recovered. The difference between the amount of carbon dioxide produced from the fermentation and the amount of carbon dioxide recovered, by weight, represents the amount of carbon dioxide collected and is measured by standard techniques. The amount of carbon dioxide collected from the fermentation may be greater than about 5, 10, 15, 20, 25, 30, 35, 40, 45, 50, 55, 60, 65, 70 75 or 80 wt % of the biogenic carbon dioxide generated during the fermentation or up to 75, 80, 85, 90, 95 or 98 wt % of the biogenic carbon dioxide generated during the fermentation. According to certain embodiments of the invention, between 5 and 85 wt %, or between 15 and 80 wt % of the biogenic carbon dioxide generated during fermentation is collected. In further embodiments of the invention, between 5 and 90 wt % or between 30 and 90 wt % of the biogenic carbon dioxide generated during the fermentation is collected. Preferably, during collection, the biogenic carbon dioxide is purified and compressed. The purification may remove water, the fuel or fuel intermediate and optionally other impurities.

(a) Collection of Biogenic Carbon Dioxide from Liquid Fuel Fermentation

Known techniques for collecting carbon dioxide from fermentations that produce liquid fuels or fuel intermediates that can be used in the practice of the invention include systems that comprise a water scrubbing unit in which water is flowed counter-current to the carbon dioxide to remove water and water soluble components, including ethanol. Water that remains in the carbon dioxide is subsequently removed in a compressor to increase the pressure of the carbon dioxide up to the water condensation level. The carbon dioxide may be fed to a drying unit to remove additional water. A purifying unit, which typically contains activated carbon, may be included in the process configuration before or after the drying unit to remove impurities. Inert gases, such as oxygen and nitrogen (also referred to in the art as non-condensable or permanent gases), may subsequently be removed in a condenser.

Although recovery of inert gases in a condenser is described, other methods can be used to remove the inert gases, and may result in improvements in recovery levels. Without being limiting, inert gases may be removed by a rectification column. A further technique for recovering high levels of carbon dioxide generated during fermentation that can be used in the practice of the invention is cold condensing to remove non-condensable gases, which relies on low temperature to decrease the solubility of the non-condensable gases so that they volatilize from the liquid phase. Cold condensing may be conducted after drying or purification.

It will be understood, however, that the invention is not restricted in scope to the methods described above and encompasses alternative procedures, including later-developed technologies, for collecting biogenic carbon dioxide from liquid fuel fermentations.

(b) Separation and Collection of Biogenic Carbon Dioxide from Biogas

As set forth previously, anaerobic digestion produces biogenic carbon dioxide and biogas comprising methane, also referred to as biogas derived methane, biomethane or renewable natural gas. Biogenic carbon dioxide mixed with biogas and optionally any other substances produced during anaerobic digestion may be separated from the biomethane by known or later-developed techniques. For example, such separation may comprise scrubbing, including water or solvent scrubbing, such as polyethylene glycol scrubbing. Scrubbing involves flowing biogas through a column with a water or solvent solution flowing counter-current to the biogas. Biogenic carbon dioxide and optionally other substances are separated from the biomethane by these techniques since carbon dioxide and other components are more soluble in water or the solvent than biomethane.

A further technique for separating biogenic carbon dioxide from the biomethane is pressure swing absorption, which utilizes adsorptive materials, such as zeolites and activated carbon that preferentially adsorb carbon dioxide at high pressure. When the pressure is released, the biogenic carbon dioxide desorbs.

It will be understood, however, that the invention is not restricted in scope to the methods described above and encompasses alternative procedures, including later-developed technologies, for separating biogenic carbon dioxide from biomethane.

### Use of Biogenic Carbon Dioxide in an Industrial Application

After collection, the biogenic carbon dioxide is supplied for displacement of geologic carbon dioxide at one or more sites that use carbon dioxide in an industrial application. The industrial application includes, without limitation, using carbon dioxide as an additive; a cooling agent, a processing agent, a treatment agent, or a carbon source to make fuels, chemicals or polymers. A site is any location that uses carbon dioxide in the industrial application. This may include a mobile site, such as a transportable container, or a fixed geographic location such as a bottling plant. The biogenic carbon dioxide may be supplied to the one or more sites by an apparatus such as a pipeline, or other transportation means, as discussed further below.

The use of carbon dioxide as an additive includes adding an amount of carbon dioxide to a substance so that it becomes a component thereof. For example, carbon dioxide may be used as a pH alterant and/or for carbonation. The carbon dioxide may be added to a food, a beverage or water. Preferably, the carbon dioxide is used to carbonate a beverage or water to produce a carbonated beverage. The beverage produced by carbonation may be beer, soda, fruit drinks, beer, wine or carbonated water.

When employed as a cooling agent, the carbon dioxide may be utilized as dry ice, CO₂ snow, as a refrigerant or in air conditioning. In another embodiment of the invention, the carbon dioxide is used as a cooling agent in food processing or preservation. For example, carbon dioxide may be used in meat slaughtering and processing, including cooling beef, poultry and pork.

The use of carbon dioxide as a processing agent may include using the carbon dioxide as a solvent. Its use as a solvent includes solvent extraction, a solvent for chemical reactions, dry cleaning and the production of small particles, such as in spray painting. Solvent extraction may include extracting components from foods. This includes supercritical carbon dioxide extraction, in which the conditions are such that the carbon dioxide is in the form of a supercritical fluid. Carbon dioxide may also be used as a solvent in the chemical industry, such as a solvent for chemical reactions, polymer syntheses and polymer modifications.

The carbon dioxide may be used as a treatment agent in a variety of applications. For example, the carbon dioxide may be used as a shielding gas in welding, a medium for extinguishing fires, or in greenhouses to treat plants for the purpose of increasing their growth.

Carbon dioxide may also be used as a carbon source to make fuels, fuel intermediates, chemicals or polymers. The fuel or fuel intermediate may include methanol, algal biofuel and syngas. The chemicals or polymers include carbonates and bicarbonates, including urea, salicyclic acid and polycarbonates.

Preferably, the carbon dioxide is used as an additive or a cooling agent. In one embodiment of the invention the carbon dioxide is used as an additive to carbonate a beverage or water or as a cooling agent in food processing or preservation.

### Supplying Biogenic Carbon Dioxide for Use in an Industrial Application

The carbon dioxide for use in the industrial application may be transported across land or sea using an apparatus adapted for such purpose. According to certain embodiments, the apparatus for transporting carbon dioxide is a pipeline, a container for transporting the biogenic carbon dioxide by rail, trucking, shipping, barge, or any other commercial distribution system. It should be appreciated that the biogenic carbon dioxide could be placed in the apparatus for storage prior to transportation. Furthermore, the apparatus for transporting carbon dioxide to the industrial application can be either integral or connected with or unconnected to an apparatus used to collect biogenic carbon dioxide. The biogenic carbon dioxide may be transported in gaseous or liquid form. Preferably, the biogenic carbon dioxide is transported in liquid form or supercritical form.

In a preferred embodiment, the apparatus is a pipeline, including a carbon dioxide dedicated pipeline, a commercial distribution pipeline or a fungible carbon dioxide pipeline. The pipeline may feed one or multiple sites that use carbon dioxide in an industrial application. Furthermore, plural carbon dioxide sources, including potentially anthropogenic or geologic carbon dioxide, may feed into the pipeline. It will be appreciated that when using a fungible carbon dioxide pipeline to supply the site or sites that use the carbon dioxide in an industrial application beneficial environmental impacts associated with biogenic carbon dioxide can be realized by end users under regulations and/or through contracts and the like. Thus, the withdrawal of non-biogenic carbon dioxide from the pipeline which delivers carbon dioxide to the site or sites that use carbon dioxide in an industrial application may be used to qualify for life cycle GHG reductions.

In another embodiment of the invention, a fuel production facility arranges for, or causes, a third party to supply biogenic carbon dioxide for use in the industrial application. The “fuel production facility” or “biofuel production facility” refers to any facility that produces a fuel or fuel intermediate by fermentation. As used herein, the term “arranging” or “causing”, means to bring about, either directly or indirectly, including through commercial arrangements such as a written agreement, verbal agreement or contract. Without being limiting, the third party may be an intermediary that obtains biogenic carbon dioxide from a fuel production facility and supplies it to a site or sites that use biogenic carbon dioxide in an industrial application.

Displacement of geologic carbon dioxide with biogenic carbon dioxide means that less geologic carbon dioxide is used in or supplied to one or more sites that use carbon dioxide in an industrial application than would otherwise be the case with an alternate geologic supply, as a result of the use or supply to such site or sites biogenic carbon dioxide collected from a fermentation, including a liquid fuel fermentation or anaerobic digestion. In one embodiment, displacement refers to a reduction in the use of geologic carbon dioxide at one or more sites that is otherwise available for use at one or more sites, wherein the reduction in use of geologic carbon dioxide results from (i) introducing biogenic carbon dioxide into an apparatus for transporting the biogenic carbon dioxide to one or more sites that use carbon dioxide in an industrial application; (ii) taking geologic carbon dioxide out of use at one or more sites and using biogenic carbon dioxide at the one or more sites; or both (i) and (ii). In a further embodiment of the invention, displacement results from the introduction of biogenic carbon dioxide into an apparatus for transporting carbon dioxide to one or more sites that used or are using geologic carbon dioxide. In yet further embodiments of the invention, displacement results from the supply of biogenic carbon dioxide for use in one or more sites that used or are using geologic carbon dioxide. Beneficially, this reduces life cycle GHG emissions of the fuel or fuel intermediate made from the fermentation process in which the biogenic carbon dioxide was produced.

As discussed, by using biogenic carbon dioxide at one or more sites that use carbon dioxide in an industrial application, the extraction of geologic carbon dioxide from underground can be prevented or reduced. Thus, according to a further embodiment of the invention, displacement involves preventing or reducing extraction of geologic carbon dioxide from underground reservoirs or deposits, referred to herein as “displacement of extraction of geologic carbon dioxide”.

In yet further embodiments, demand for geologic carbon dioxide is reduced due to the supply of biogenic carbon dioxide to one or more sites where this reduced demand qualifies for reducing life cycle GHG emissions. In one such embodiment, displacement results from a reduction in demand for geologic carbon dioxide that is otherwise available for use at one or more sites that use carbon dioxide in an industrial application, wherein the reduction in demand of geologic carbon dioxide results from (i) introducing biogenic carbon dioxide into an apparatus for transporting carbon dioxide to one or more sites; or (ii) taking geologic carbon dioxide out of use at one or more sites and using biogenic carbon dioxide at the one or more sites.

The use of biogenic carbon dioxide to displace geologic carbon dioxide includes supplying biogenic carbon dioxide for use, for example by another party, in replacing, substituting or using biogenic carbon dioxide as a priority over geologic carbon dioxide that could otherwise be used at the site. Preferably, the biogenic carbon dioxide supplied for use in the industrial application displaces a corresponding amount of geologic carbon dioxide used in the industrial application. This may involve taking out of use a first amount of geologic carbon dioxide at one or more sites and supplying, preferably subsequently supplying, an amount of biogenic carbon dioxide at one or more sites to displace the first amount of geologic carbon dioxide. The biogenic carbon dioxide may displace all of the geologic carbon dioxide used in the site that uses carbon dioxide in an industrial application, or a portion thereof.

By way of example, if 10 units of biogenic carbon dioxide are introduced to a pipeline and 10 units of carbon dioxide are withdrawn from the pipeline with the GHG emission attributes of the input biogenic carbon dioxide and used in a site, and 10 units of geologic carbon dioxide are taken out of use or removed from use at the site, then 10 units of geologic carbon dioxide have been displaced at the site. It should be understood that the biogenic carbon dioxide may displace only a portion of the geologic carbon dioxide used in the site. For example, if a site previously used 100 units of geologic carbon dioxide and 10 units of the geologic carbon dioxide are displaced by 10 units of biogenic carbon dioxide, then only 90 units of geologic carbon dioxide need be used in the site. Additionally, displacement may occur if biogenic carbon dioxide is used to increase the amount of carbon dioxide used at the site. For example, if the site previously used 100 units of geologic carbon dioxide and an additional 10 units of biogenic carbon dioxide are then used at the site, so that 110 units of carbon dioxide are used, then 10 units of geologic carbon dioxide can be considered displaced because the demand for an additional 10 units of geologic carbon dioxide has been obviated.

When the biogenic carbon dioxide is transported by pipeline, the amount of geologic carbon dioxide that is displaced by biogenic carbon dioxide may be measured by gas metering. For example, a meter would be placed in proximity to the facility in which biogenic carbon dioxide is produced to measure the amount of biogenic carbon dioxide supplied to the pipeline. Similarly, the amount of carbon dioxide withdrawn from the pipeline for supply to the site would be metered. Written documentation as described herein can set out the amounts of biogenic carbon dioxide introduced to the pipeline and withdrawn for use at the site. Optionally, the producer of biogenic carbon dioxide would contract with the owner of a site to supply biogenic carbon dioxide. If the pipeline is supplied by multiple carbon dioxide sources, some of which are non-biogenic, the carbon dioxide withdrawn may be non-biogenic or contain a mixture of biogenic and non-biogenic carbon dioxide. Nonetheless, an amount of carbon dioxide equal to the input amount of biogenic carbon dioxide can be withdrawn and can qualify for life cycle GHG reductions, for the reasons discussed previously.

In one embodiment, the introduction of biogenic carbon dioxide into an apparatus for transporting carbon dioxide to one or more sites that used or are using geologic carbon dioxide reduces GHG emissions because the biogenic carbon dioxide performs the function previously carried out by geologic carbon dioxide. According to a preferred embodiment of the invention, such a displacement can be evidenced by written documentation that sets out a life cycle analysis of the fermentation based fuel and includes in the analysis a GHG emission reduction calculation due to a displacement of geologic carbon dioxide that could be used in the absence of the use of biogenic carbon dioxide at the site. In some embodiments, the GHG emission reductions occur even in situations where there is no immediate reduction in the use of geologic carbon dioxide. Notably, this concept is similar to indirect land use impacts on greenhouse gas emissions, which are commonly used in life cycle GHG emission analyses of biofuels. In the case of land use impacts, changes in emission are calculated using the lifetime emissions effects associated with forecast changes in long term land use.

Over the lifetime of a site or sites using geologic carbon dioxide, use of biogenic carbon dioxide may lead to avoided use of geologic carbon dioxide even if there is no immediate reduction in the use of geologic carbon dioxide at the site. By “lifetime of the site or sites”, it is meant the time period from which carbon dioxide is first used at a site up until the last use occurs prior to permanent closure of the site. In certain embodiments of the invention, over the lifetime of a site employing carbon dioxide, there may be a finite amount of carbon dioxide that is used. Because the total carbon dioxide use is finite, when biogenic carbon dioxide is used at the site, there is a reduced amount of total geologic carbon dioxide used. In this case, a displacement of geologic carbon dioxide by biogenic carbon dioxide occurs over the lifetime of a site or sites, and such displacement is considered to provide GHG emissions benefits over the lifetime of the site or sites, even if there is not an immediate reduction in the use of geologic carbon dioxide.

According to some embodiments of the invention, such GHG emission reductions are quantified in data and written documentation including, but not limited to, a letter, memorandum, affidavit, form or submission to governmental authorities or a contract that states, commits, guarantees or otherwise indicates that biogenic carbon dioxide is used to displace the use of geologic carbon dioxide. The written documentation may, for example, comprise data describing a life cycle GHG analysis which includes a quantification of a GHG emission reduction of the fuel or fuel intermediate due to a reduction in the use of geologic carbon dioxide that would occur over the lifetime of the site or sites as a result of the use of biogenic carbon dioxide. Written documentation or data evidencing such a displacement that reduces GHG emissions is typically supplied to, and meets the requirements of, government regulators, such as the EPA.

According to further embodiments of the present invention, in order to determine that the biogenic carbon dioxide is being used to displace geologic carbon dioxide, at least one or more of the sites meets at least two of the conditions selected from: (a) the site has used geologic carbon dioxide in the industrial application; (b) the site has access to geologic carbon dioxide for use in the industrial application; and (c) written documentation indicates that biogenic carbon dioxide is being used to displace geologic carbon dioxide.

Referring to condition (a), “used geologic carbon dioxide in the industrial application”, means that the site has used geologic carbon dioxide in the industrial application at some time in its history, including but not limited to times prior to or after the start of its use of the biogenic carbon dioxide. It should be appreciated that the time span between use of geologic carbon dioxide and the subsequent use of the biogenic carbon dioxide can vary. That is, the geologic carbon dioxide can be taken out of use, and immediately followed by the use of biogenic carbon dioxide or the period of time between geologic and biogenic carbon dioxide use can span a longer period of time, for example, days, months or even years. Furthermore, it should be understood that there could be some intermixing of geologic, anthropogenic and biogenic carbon dioxide in the industrial application.

Access to geologic carbon dioxide refers to the ability to use geologic carbon dioxide at the site if biogenic carbon dioxide were not available. A site has access to geologic carbon dioxide if, for example, it is or was served by a pipeline that delivered geologic carbon dioxide. In some embodiments of the invention, a site has access to geologic carbon dioxide by being located within a 100 mile radius from the closest point on a carbon dioxide pipeline into which geologic carbon dioxide is fed or within a 75, 50, 25 or 10 mile radius. According to a further embodiment, the carbon dioxide pipeline into which geologic carbon dioxide is fed either is presently connected to the site or was connected to the site in the past. Preferably, the pipeline is connected to the site.

As discussed, displacement in accordance with the invention may be evidenced by data and written documentation, which indicates that biogenic carbon dioxide is being used to displace geologic carbon dioxide at the site, as set out below. To attain credit for life cycle carbon dioxide emissions reductions, written documentation is generated which contains a life cycle GHG analysis of the fermentation fuel or fuel intermediate that includes displacement of geologic carbon dioxide as contributing in whole or in part to life cycle GHG reduction. Such documentation is typically supplied to a government regulatory authority.

By “written documentation indicates that biogenic carbon dioxide is being used to displace geologic carbon dioxide” at the site, it is meant that a written document including, but not limited to, a letter, memorandum, affidavit, form or submission to governmental authorities or a contract states, commits, guarantees or otherwise indicates that biogenic carbon dioxide is used to displace, replace, substitute for or otherwise reduce the use of geologic carbon dioxide. The written documentation may comprise data or documentation describing a life cycle GHG analysis indicating that the use or supply of biogenic carbon dioxide for displacement of geologic carbon dioxide creates a net GHG benefit.

In another embodiment, there is provided a process that comprises fermenting organic material to produce a fermentation based fuel, or fuel intermediate, such as an alcohol, collecting at least 5 wt % of the biogenic carbon dioxide that is produced in the fermentation, introducing all or a portion of the biogenic carbon dioxide into apparatus for transporting the biogenic carbon dioxide to one or more sites that use carbon dioxide in an industrial application, wherein, in respect to one or more of the sites, written documentation indicates that the use of biogenic carbon dioxide to displace geologic carbon dioxide is included in a net life cycle carbon dioxide emissions analysis. This life cycle carbon dioxide emissions analysis includes a carbon dioxide emissions savings due in whole or in part from the supply of biogenic carbon dioxide.

In a further embodiment, there is provided a process that comprises: fermenting the organic material to produce a fuel or fuel intermediate and biogenic carbon dioxide; collecting an amount of biogenic carbon dioxide, for example at least 5 wt % of the biogenic carbon dioxide generated from the step of fermenting; introducing the biogenic carbon dioxide into apparatus for transporting the biogenic carbon dioxide to one or more sites that use carbon dioxide in an industrial application, wherein at least one of the sites meets one of the following conditions: (a) the site has used geologic carbon dioxide in the industrial application; and (b) the site has access to geologic carbon dioxide for use in the industrial application; and supplying the biogenic carbon dioxide for use in one or more sites to displace geologic carbon dioxide, as evidenced by written documentation. The written documentation indicates that biogenic carbon dioxide is being used to displace geologic carbon dioxide at the site, as set out above.

Additional methods can be employed in combination with displacing geologic carbon dioxide with biogenic carbon dioxide to reduce the overall GHG life cycle emissions of the fuel or fuel intermediate. Such methods include, without limitation, increasing energy efficiency, energy saving and fuel switching. For example, the energy efficiency of a fermentation fuel production facility can be improved by, for example, increasing the number of stages of evaporation and distillation, employing heat recovery on dryers or using combined heat and power generation. Energy requirements can be lessened by reducing or eliminating energy consuming operations such as the drying of distillers grains. Fuel switching can reduce life cycle emissions by, for example, replacing natural gas, a fossil fuel, with biogas, a renewable fuel. Thus, even if a relatively small amount of the biogenic carbon dioxide generated in the fermentation is collected and supplied to one or more sites for displacement of geologic carbon dioxide, the life cycle GHG emission reduction of the fuel or fuel intermediate relative to the gasoline baseline may still meet the threshold to generate a desired fuel credit if one or more of these additional methods are employed in combination with the invention. For example, if the life cycle GHG emissions of a fuel or fuel intermediate are reduced by one or more other methods, a life cycle GHG emission reduction of 50% relative to a gasoline baseline for a particular fuel or fuel intermediate could still be achieved if 5 wt % of the carbon dioxide produced from a fermentation is collected and used to displace geologic carbon dioxide at a site.

### Use of the Fuel or Fuel Intermediate

The fermentation based fuel of the invention can be used as a transportation fuel. For example, ethanol may be blended with gasoline at levels from 5% to 85% ethanol and used to power motor vehicles. Ethanol is typically recovered by distillation and an azeotropic breaking process prior to blending with gasoline. Ethanol can alternatively be used as a feedstock for making a transportation fuel component such as ethyl tert-butyl ether. Biogas derived methane may be used directly to power vehicles, or used as a feedstock to make transportation fuel, for example as disclosed in co-pending U.S. non-provisional application Ser. No. 13/721,157, which is incorporated herein by reference in its entirety.

Alternatively, the fermentation based fuel can be used as an energy source for heating or to produce electricity. For example, biomethane or methane having reduced GHG emissions due to implementation of the invention can be used to supply energy to a fuel production facility, which includes any operation that produces a fuel or fuel intermediate from organic material, such as a liquid fuel production facility, including an ethanol production facility. The methane can also supply energy to any equipment used to support a fuel production process in a fuel production facility, referred to herein as “associated utilities”. The methane in this embodiment is biogas derived methane (also referred to as biomethane), including methane that qualifies under applicable laws or regulations as being renewably derived, as set forth below. In one embodiment, such methane is used in any part of a fuel production facility or associated utilities to supply heat and/or electricity. The methane can be combusted to provide steam, which can be used to drive turbines to create electricity for plant needs and/or to supply thermal energy within the facility. The methane can also be used in a direct gas turbine to make electricity. Thermal energy can be used for on-site heating, as process heat or for cooling operations. Furthermore, if electricity is generated from the methane, heat that is produced as a by-product during the electricity generation can often be used in the facility.

It should be appreciated that some of the methane used to supply energy to a fuel production facility or associated utilities can be natural gas. In other words, the energy need not be supplied exclusively by biomethane, but can be a combination of both natural gas and biomethane.

Biomethane can be transported to the fuel production facility by any suitable apparatus for transporting methane to a fuel production facility. In a preferred embodiment, such apparatus will be a pipeline, such as a natural gas pipeline or a biogas dedicated pipeline. Alternatively, the apparatus may be a container for transporting the biomethane by rail, trucking or shipping, or any other commercial distribution system.

In a preferred embodiment, the biomethane will be transported via a pipeline. If the pipeline is fed by a plurality of methane sources, some of which are not sourced from biomethane, the methane withdrawn may not contain actual molecules from the original organic material from which the biomethane is derived, but rather the energy equivalent value of the biomethane. With respect to biomethane used for electricity generation in a facility, government authorities have recognized that it does not make any difference, in terms of the beneficial environmental attributes associated with the use of biomethane, whether the displacement of fossil fuel occurs in a fungible natural gas pipeline, or in a specific fuel production facility that draws methane from that pipeline. Thus, methane withdrawn from a pipeline that is fed by biomethane, as well as methane derived from sources besides biomethane, will still be considered biomethane or biogas derived methane. As would be appreciated by those of skill in the art, the amount of methane withdrawn from such a pipeline with the GHG emission attributes of biomethane and the amount of biomethane fed to the pipeline will typically be consistent. The amount of biomethane fed to the pipeline and the amount of methane withdrawn can be determined by gas metering.

The methane produced using the invention that is supplied to the fuel production facility to provide energy has reduced life cycle GHG emissions. The reduced life cycle GHG emissions are measured relative to the biomethane production process baseline. Biomethane or methane that has reduced life cycle GHG emissions relative to this production process baseline is also referred to herein as “enhanced GHG biomethane”. As set forth previously, a biomethane production process baseline refers to the life cycle GHG emissions associated with a biogas production process conducted under identical conditions except the biogenic carbon dioxide that is separated from the biomethane is released to the atmosphere. In some embodiments of the invention, the reduction in life cycle GHG emissions results in whole or in part from the practice of or arranging for the practice of the following process by one or more third parties: (i) anaerobically digesting organic material to produce biogas comprising biomethane and biogenic carbon dioxide; (ii) separating the biomethane and biogenic carbon dioxide; (iii) collecting an amount of the biogenic carbon dioxide generated from the step of separating; and (iv) supplying the biogenic carbon dioxide from step (iii) for use in one or more sites that use carbon dioxide in an industrial application for displacement of geologic carbon dioxide.

By arranging for the practice of the foregoing process by one or more third parties, it is meant to bring about the process, either directly or indirectly, including through commercial arrangements such as a written agreement, verbal agreement or contract.

Advantageously, when a fuel production facility receives and uses such enhanced GHG biomethane, the life cycle GHG or carbon dioxide emissions of the liquid fuel or fuel intermediate produced in the facility can be reduced significantly relative to a gasoline baseline.

Thus, according to certain aspects of the invention, there is provided a process to reduce the life cycle GHG or carbon dioxide emissions associated with production of a liquid fuel or fuel intermediate, the process comprising: (i) producing sugar from plant derived organic material and converting the sugar to the liquid fuel or fuel intermediate in a fuel production facility; (ii) using methane to supply energy in any part of the fuel production facility or associated utilities, wherein the methane has associated with it life cycle GHG or carbon dioxide emissions that are reduced relative to a biomethane production process baseline due to or as a result of the practice of or arranging for the practice of the following process by one or more third parties: (a) the collection of biogenic carbon dioxide from biogas comprising biomethane; (b) the supply of biogenic carbon dioxide collected in step (a) to one or more sites that use carbon dioxide in an industrial application; (c) the introduction of the biomethane into an apparatus for transporting to the fuel production facility; and (d) the withdrawal of methane from the apparatus to supply energy in any part of the fuel production facility or associated utilities.

In another embodiment, there is provided a process to reduce the life cycle GHG or carbon dioxide emissions associated with production of a liquid fuel or fuel intermediate, the process comprising: (i) producing sugar from plant derived organic material and converting the sugar to the liquid fuel or fuel intermediate in a fuel production facility; (ii) using methane to supply energy in any part of the fuel production facility or associated utilities, wherein the methane has associated with it life cycle GHG or carbon dioxide emissions that are reduced relative to a biomethane production process baseline where such reduction is due in whole or in part to the displacement of geologic carbon dioxide with biogenic carbon dioxide that originated from an anaerobic digestion that produces biogas comprising biomethane and biogenic carbon dioxide.

According to a further embodiment of the invention, an amount of biogenic carbon dioxide that is produced from the above-mentioned step of converting the sugar to the liquid fuel or fuel intermediate is collected and supplied for use in one or more sites that use carbon dioxide in an industrial application for displacement of geological carbon dioxide. The GHG emission reductions from collecting carbon dioxide from the conversion and using it to displace geologic carbon dioxide in an industrial application using carbon dioxide, combined with those resulting from using the methane having reduced GHG emissions in the fuel production facility to generate energy, may reduce the overall life cycle GHG emissions of the fuel or fuel intermediate relative to the gasoline baseline to a level that meets the threshold to generate a desired fuel credit.

The process may further comprise generating a renewable fuel credit associated with the liquid fuel or fuel intermediate. In some embodiments of the invention, the fuel credit is a RIN or an LCFS credit. The foregoing process may allow the fuel or fuel intermediate produced by the facility to qualify for a RIN having higher market value or for the generation of more LCFS credits, or both. If a RIN is generated, it is preferably a D5 RIN or a D3 RIN.

### Measuring Life Cycle GHG Emissions of Fermentation Based Fuel

As discussed, the GHG emission reductions realized by the invention allow fuel production facilities, or other parties, to qualify for more desirable renewable fuel credits than could otherwise be attained.

Prior to generating the renewable fuel credit, a party generates data representative of a life cycle GHG emission reduction of the liquid fuel or fuel intermediate relative to a gasoline baseline. The party may be the party that carries out the fermentation to produce the liquid fuel or fuel intermediate, i.e., a fuel production facility, or a third party. Such third party may be a regulatory body, such as the EPA. The data representative of a life cycle GHG emission reduction may be a percent reduction in GHG emissions (typically measured in CO₂ equivalents) of a fuel relative to a gasoline baseline.

The amount of carbon dioxide savings for the fuel or fuel intermediate can be calculated using methods known in the art. As much as 36.12 g CO₂ eq/MJ of ethanol (38,145 g CO₂ eq/MMBTU) can be obtained from collecting and using 100 wt % of the carbon dioxide evolved in fermentation and using it in accordance with the invention. This assumes no residual losses of carbon dioxide in collection, purification and transportation.

The ultimate amount of reduction in life cycle carbon dioxide emissions will depend on the type of fuel, fuel intermediate or alcohol produced, which influences the stoichiometry of the fermentation reaction, the amount of carbon dioxide collected and also any carbon dioxide losses associated with the process, e.g., in collection, purification, compression and transport. Without being limiting, it has been reported that as much as 80 wt % of carbon dioxide evolved from ethanol fermentation can be recovered (Buchhauser U. et al., 2008, CO₂ Recovery: Improved Performance with a Newly Developed System, MBAA Technical Quarterly, 45(1):84-89). By way of example, if 38,145 g CO₂ eq/MMBTU is evolved in fermentation and 80 wt % of the amount of carbon dioxide evolved in fermentation is collected from a sorghum ethanol process with GHG fluxes comparable to Table 7 (see Example 1(b)), then the life cycle GHG emissions change associated with displacement of the geologic carbon dioxide by the biogenic carbon dioxide will lead to a reduction in the life cycle GHG emissions associated with the production of ethanol of approximately 23,308 g CO₂ eq/MMBTU ethanol relative to a production process baseline, taking into account 7,207 g CO₂ eq/MMBTU emissions due to the collection, purification, compression and transport of biogenic carbon dioxide.

It should be understood that the upper limit of carbon dioxide that is recovered and the losses due to collection, purification, compression and transport are exemplary and should not be construed to limit the current invention in any manner. For instance, as much as 36.12 g CO₂ eq/MJ of ethanol (38,145 g CO₂ eq/MMBTU) can be obtained from collecting and using 100 wt % of the carbon dioxide evolved in fermentation and using it in accordance with the invention, although this assumes no residual losses of carbon dioxide in collection, purification and transportation. According to certain embodiments, the invention reduces the life cycle GHG emissions associated with the production of a fuel or fuel intermediate, by between about 1.0 CO₂ eq/MJ and about 50 CO₂ eq/MJ, or between about 1.0 g CO₂ eq/MJ and about 40 g CO₂ eq/MJ, or between about 1.0 g CO₂ eq/MJ and 30 g CO₂ eq/MJ, or between about 1.0 g CO₂ eq/MJ and 25 g CO₂ eq/MJ, or between about 2.0 g CO₂ eq/MJ and about 25 g CO₂ eq/MJ, or between about 5.0 g CO₂ eq/MJ and about 25 g CO₂ eq/MJ or between about 5.0 g CO₂ eq/MJ and about 22.5 g CO₂ eq/MJ relative to a production process baseline.

According to further embodiments, the fuel produced by the fermentation is ethanol and the invention reduces the carbon dioxide or life cycle GHG emissions associated with the production of the ethanol by between about 1.0 g CO₂ eq/MJ and about 35 g CO₂ eq/MJ, or between about 2.0 g CO₂ eq/MJ and about 35 g CO₂ eq/MJ, or between about 2.0 g CO₂ eq/MJ and about 25 g CO₂ eq/MJ, or between about 5.0 g CO₂ eq/MJ and about 25 g CO₂ eq/MJ or between about 10 g CO₂ eq/MJ and about 25 g CO₂ eq/MJ relative to a production process baseline.

According to other embodiments, the fuel produced by the fermentation is butanol and the invention reduces the life cycle GHG emissions associated with the production of the butanol by between about 1.0 g CO₂ eq/MJ and about 35 g CO₂ eq/MJ, or between about 2 g CO₂ eq/MJ and about 35 g CO₂ eq/MJ or between about 2.0 g CO₂ eq/MJ and about 25 g CO₂ eq/MJ, or between about 5.0 g CO₂ eq/MJ and about 25 g CO₂ eq/MJ or between about 10 g CO₂ eq/MJ and about 25 g CO₂ eq/MJ relative to a production process baseline.

According to other embodiments, the fuel produced by the fermentation is biomethane and the invention reduces the life cycle GHG emissions associated with the production of the methane by between about 1.0 g CO₂ eq/MJ and about 50 g CO₂ eq/MJ, or between about 2.0 g CO₂ eq/MJ and about 45 g CO₂ eq/MJ, or between about 5.0 g CO₂ eq/MJ and about 40 g CO₂ eq/MJ, or between about 5.0 g CO₂ eq/MJ and about 35 g CO₂ eq/MJ, or between about 10 g CO₂ eq/MJ and about 35 g CO₂ eq/MJ relative to a biomethane production process baseline.

According to further embodiments, the invention reduces the life cycle GHG emissions associated with the production of ethanol or butanol by at least 1.0, 2.0, 3.0, 4.0, 5.0, 6.0, 7.0, 8.0, 9.0, 10, 11, 12, 13, 14, 15, 16, 17, 18, 19 or 20 g CO₂ eq/MJ relative to a production process baseline. According to other embodiments, the invention reduces the life cycle GHG emissions associated with the production of ethanol or butanol by up to 20, 21, 22, 23, 24, 25, 26, 27, 28, 29, 30, 31, 32, 33, 34 or 35 g CO₂ eq/MJ relative to a production process baseline.

According to further embodiments, the invention reduces the life cycle GHG emissions associated with the production of biomethane by at least 1.0, 2.0, 3.0, 4.0, 5.0, 6.0, 7.0, 8.0, 9.0, 10, 11, 12, 13, 14, 15, 16, 17, 18, 19 or 20 g CO₂ eq/MJ relative to a biomethane production process baseline. According to other embodiments, the invention reduces the life cycle GHG emissions associated with the production of biomethane by up to 25, 26, 27, 28, 29, 30, 31, 32, 33, 34, 35, 36, 37, 38, 39, 40, 41, 42, 43, 44, 45, 46, 47, 48, 49 or 50 g CO₂ eq/MJ relative to a biomethane production process baseline.

Furthermore, any units of g CO₂ eq/MJ or CO₂/MJ provided herein can be converted to g CO₂ eq/MMBTU or CO₂/MMBTU by multiplying by a conversion factor of 1054.35. Similarly, any units of CO₂ eq/MMBTU or CO₂/MMBTU can be converted to CO₂ eq/MJ or CO₂/MJ by dividing by this conversion factor.

According to certain particularly advantageous embodiments, the present invention provides a process to reduce the life cycle GHG emissions associated with the production of a fermentation based fuel, including ethanol for use as a fuel or fuel intermediate, by approximately 9.0 g CO₂ eq/MJ (about 9,536 g CO₂ eq/MMBTU) or more relative to a production process baseline based upon the capture of approximately 25 wt % or more of the amount of biogenic carbon dioxide evolved in fermentation. Preferably, the reduction in life cycle GHG emissions is greater than 20 g CO₂ eq/MJ (about 21,100 g CO₂ eq/MMBTU) relative to a production process baseline, and wherein at least approximately 55 wt % or more of biogenic carbon dioxide evolved in fermentation is captured and the fermentation based fuel is an alcohol that is ethanol, butanol or other isomer of butanol such as isobutanol.

According to further embodiments of the invention, the present invention provides a process to reduce the life cycle GHG emissions associated with the production of a fermentation based fuel, including alcohol used as a fuel or fuel intermediate, in which the amount of biogenic carbon dioxide used in an industrial application leads or contributes to an overall life cycle GHG emissions level that is less than 50% that of a gasoline baseline. Preferably, the fermentation based fuel is an alcohol, such as ethanol produced by the fermentation of wheat or sorghum, or butanol, including butanol isomers, such as isobutanol, produced from corn starch. In such an embodiment, the ethanol or butanol so produced would qualify for generation of a D5 RIN, as discussed hereinafter.

To determine life cycle GHG emissions associated with the fermentation based fuel or alcohol of the present invention, analyses are conducted to calculate the GHG emissions related to the production and use of the fermentation based fuel or alcohol throughout its life cycle. Life cycle GHG emissions include the aggregate quantity of GHG emissions related to the full life cycle of the fermentation based fuel or alcohol, including all stages of fuel and feedstock production and distribution, from feedstock generation or extraction through the distribution and delivery and use of the finished fuel to the ultimate consumer. GHG emissions account for total net GHG emissions, both direct and indirect, associated with feedstock production and distribution, the fuel and fuel intermediate production and distribution and use.

Because many of the laws adopted differentiate the requirements for fuels based upon their net GHG emissions impacts, it is known to those skilled in the art that regulators have developed and/or adopted methods to analyze and characterize the expected net GHG emissions of fuel pathways. Thus, according to certain embodiments of the invention, life cycle GHG emissions are determined in accordance with prevailing rules and regulations.

Life cycle GHG emissions evaluations generally consider GHG emissions associated with each of:
(a) feedstock production and recovery, including the source of carbon dioxide in the feedstock, direct impacts such as chemical inputs, energy inputs, and emissions from the collection and recovery operations, and indirect impacts such as the impact of land use changes from incremental feedstock production;
(b) feedstock transport, including feedstock production and recovery GHG emissions from feedstock transport including energy inputs and emissions from transport;
(c) fuel production, including chemical and energy inputs, emissions and byproducts from fuel production (including direct and indirect impacts); and
(d) transport and storage of the fuel prior to use as a transport or heating fuel, including chemical and energy inputs and emissions from transport and storage.

Examples of models to measure life cycle GHG emissions associated with the production of a fermentation based fuel, such as an alcohol, include, but are not limited to:
(i) GREET Model—GHGs, Regulated Emissions, and Energy Use in Transportation, the spread-sheet analysis tool developed by Argonne National Laboratories;
(ii) FASOM Model—a partial equilibrium economic model of the U.S. forest and agricultural sectors developed by Texas A&M University;
(iii) FAPRI International Model—a worldwide agricultural sector economic model that was run by the Center for Agricultural and Rural Development (“CARD”) at Iowa State University;
(iv) GTAP Model—the Global Trade Analysis Project model, a multi-region, multi-sector computable general equilibrium model that estimates changes in world agricultural production as well as multiple additional models; and
(v) ISO (International Organization for Standardization) standards for GHG emissions accounting and verification—provides guidance for quantification, monitoring and reporting of activities intended to cause greenhouse gas (GHG) emission reductions or removal enhancements.

One benefit of the present invention is the ability to create co-product credits. Co-product credits can be assigned if a co-product is produced in a biofuel production facility. The co-product displaces equivalent products in the market produced from fossil fuel energy sources. This reduces GHG emissions because fossil fuel energy to produce the equivalent co-product by conventional methods is reduced. With respect to the invention, the biogenic carbon dioxide displaces the use of geologic carbon dioxide and this substitutes carbon dioxide of underground origin by carbon dioxide from atmospheric origin, thereby improving atmospheric carbon dioxide levels. Examples of methodologies for calculating GHG emissions, or carbon intensity, that take into account co-product credits are disclosed in Detailed California-Modified GREET Pathway for Corn Ethanol, California Environmental Protection Agency, Air Resources Board, Jan. 20, 2009, Version 2.0; Wang et al., 2011, Energy Policy 39:5726-5736; and White Paper, Issues Related to Accounting for Co-Product Credits in the California Low Carbon Fuel Standard, State of California, Air Resources Board, each of which is incorporated herein by reference.

The life cycle GHG emissions or carbon intensity of the fuel or fuel intermediate of the invention are measured in carbon dioxide equivalents (CO₂ eq). As would be understood by those of skill in the art, carbon dioxide equivalents are used to compare the emissions from various GHGs based upon their global warming potential (GWP), which is a conversion factor that varies depending on the gas. The carbon dioxide equivalent for a gas is derived by multiplying the amount of the gas by the associated GWP.

grams of CO₂eq=((grams of a gas)*(GWP of the gas))

The GWP conversion value used to determine g CO₂ eq will depend on applicable regulations for calculating life cycle GHG emissions reductions. The GWP under EISA is 1, 21 and 310, respectively, for carbon dioxide, methane and nitrous oxide as set forth in Renewable Fuel Standard Program (RFS2) Regulatory Impact Analysis, February 2010, United States Environmental Protection Agency, EPA-420-R-10-006, pg. 13, of which the entire contents are incorporated herein by reference. Under California's LCFS, the GWP is 1, 25 and 298, respectively, for carbon dioxide, methane and nitrous oxide, as measured by the GREET model.

The unit of measure for carbon intensity or life cycle GHG emissions that may be used to quantify GHG emissions of the fuel or fuel intermediate of the present invention is grams CO₂ eq per MJ of energy in the fuel or grams CO₂ eq per million British thermal units of energy in the fuel (MMBTU). The units used to measure life cycle GHG emissions will generally depend on applicable regulations. For example, under the EPA regulations, GHG emissions are measured in units of grams CO₂ eq per million BTUs (MMBTU) of energy in the fuel. Under LCFS, GHG emissions are measured in units of grams CO₂ eq per MJ of energy in the fuel and are referred to as “carbon intensity” or “CI”. The life cycle GHG emissions of the renewable fuel are compared to the life cycle GHG emissions for gasoline, referred to as a gasoline baseline. GHG life cycle emissions are compared by reference to the use of gasoline per unit of fuel energy. The value of the gasoline baseline used in life cycle GHG emission calculations can depend on the regulatory body. The EPA measures the carbon intensity of gasoline (gasoline baseline) as 98,204 g CO₂ eq/MMBTU or 93.10 g CO₂ eq/MJ. Under California's LCFS, the gasoline baseline is 95.86 g CO₂ eq/MJ. Those of ordinary skill in the art can readily convert values herein from g CO₂ eq/MJ to g CO₂ eq/MMBTU or g CO₂ eq/MMBTU to g CO₂ eq/MJ by using an appropriate conversion factor. Further, it should be appreciated that the value of the gasoline baseline can change from time to time depending on prevailing regulations.

According to certain embodiments of the invention, the life cycle GHG emission reduction relative to a gasoline baseline is measured “using EPA methodology”, which means measuring life cycle GHG emissions reductions as disclosed in EPA-420-R-10-006 (supra), or supplanted by prevailing methodologies used by the EPA, which are publicly available.

According to a further embodiment of the invention, the life cycle GHG emission reduction relative to a gasoline baseline is measured using “LCFS methodology”, which means measuring life cycle GHG emissions reductions by California's LCFS methodology using the GREET model, as set forth in Detailed California-Modified GREET Pathway for Corn Ethanol, supra, or supplanted by prevailing methodologies used by regulators, which are publicly available.

According to one embodiment of the invention, the life cycle carbon dioxide emissions, rather than the life cycle GHG emissions, are determined for the fuel or fuel intermediate and compared to a gasoline baseline. For example, in those embodiments in which a reduction in carbon dioxide emissions relative to a production process baseline is quantified, a life cycle carbon dioxide emission reduction can be quantified instead of a life cycle GHG emission reduction.

### Meeting Renewable and Low Carbon Fuel Targets

As mentioned, in view of the life cycle GHG savings that are achievable by the present invention, the fuel or fuel intermediate of the invention can qualify for a renewable fuel credit that has higher market value than other renewable fuel credits associated with lower life cycle GHG savings thresholds. For example, the fuel or fuel intermediate of the invention may have a life cycle GHG emission reduction of 50% or more relative to a gasoline baseline, and thus could qualify for a RIN under EISA having a D code of 5, which is an advanced biofuel under current regulations. A RIN having a D code of 5 has a higher market value than other RINs, such as a RIN having a D code of 6 requiring only a life cycle GHG emission reduction of 20% relative to a gasoline baseline under current regulations. Likewise, under the LCFS, fuels with greater reductions in life cycle GHG emissions qualify for a greater number of credits having higher market value than fuels with lower reductions. According to some embodiments of the invention, the fuel qualifies for both higher market value RINs and a greater number of credits under LCFS.

The credit may be generated by a fuel production facility or any other party in possession of the fermentation based fuel or fuel intermediate. This may include an intermediary party that provides the fermentation based fuel to a fuel blender or importer, or the fuel blender or importer themselves. According to certain embodiments of the invention, the credit or renewable fuel credit is caused to be generated by another party. According to such embodiments, a producer of the fermentation based fuel or fuel intermediate may cause an intermediary or other party, including a fuel blender or importer, to generate a credit.

Energy policy, including EISA and LCFS, and the generation of renewable fuel credits under each of these legislative frameworks, is discussed in turn below.

### (i) Meeting Renewable Fuel Targets Under EISA

U.S. policymakers have introduced a combination of policies to support the production and consumption of biofuels and one important element of U.S. biofuel policy is the RFS. The RFS originated with the Energy Policy Act of 2005 (known as RFS1) and was expanded and extended by the EISA of 2007. The RFS expanded and extended under EISA is sometimes referred to as RFS2 or RFS as used herein.

Under the EISA, the RFS sets annual mandates for renewable fuels sold or introduced into commerce in the United States. The RFS sets mandates through 2022 for different categories of biofuels (see Table 3 below). There is an annually increasing schedule for minimum aggregate use of total renewable biofuel (comprised of conventional biofuels and advanced biofuels), total advanced biofuel (comprised of cellulosic biofuels, biomass-based diesel, and other advanced biofuels), cellulosic biofuel and bio-based diesel. The RFS mandates are prorated down to “obligated parties”, including individual gasoline and diesel producers and/or importers, based on their annual production and/or imports.

Each year, obligated parties are required to meet their prorated share of the RFS mandates by accumulating credits known as RINs, either through blending designated quantities of different categories of biofuels, or by purchasing from others the RINs of the required biofuel categories.

The RIN system was created by the EPA to facilitate compliance with the RFS. Credits called RINs are used as a currency for credit trading and compliance. RINs are generated by producers and importers of renewable biofuels and assigned to the volumes of renewable biofuels transferred into the fuel pool. RINs are transferred with the renewable fuel through the distribution system until they are separated from the fuel by parties who are entitled to make such separation (generally refiners, importers, or parties that blend renewable fuels into finished fuels). After separation, RINs may be used for RFS compliance, held for future compliance, or traded. There is a centralized trading system administered by the U.S. EPA to manage the recording and transfer of all RINs.

As would be appreciated by those of skill in the art, a RIN generated prior to Jul. 1, 2010 was a 38 character numeric code that corresponded to a volume of renewable fuel produced in or imported into the United States. According to certain embodiments of the invention, a RIN may be characterized as numerical information. The RIN numbering system was in the format KYYYYCCCCFFFFFBBBBBRRDSSSSSSSSEEEEEEEE where numbers are used to designate a code representing whether the RIN is separated from or attached to a specific volume (K), the calendar year of production or import (YYYY), Company ID (CCCC), Facility ID (FFFFF), Batch Number (BBBBB), a code for fuel equivalence value of the fuel (RR), a code for the renewable fuel category (D), the start of the RIN block (SSSSSSSS) and the end of the RIN block (EEEEEEEE). It should be appreciated that the information required for RIN generation and/or the format of the information may change depending on prevailing regulations. Under current regulations, a RIN contains much of the foregoing information and other information in the form of data elements that are introduced into a web-based system administered by the EPA known as the EPA Moderated Transaction System, or “EMTS”.

The D code of a RIN specifies the fuel type, feedstock and production process requirements and thus in certain embodiments of the invention the D code may be used to characterize the type of RIN, as set out hereinafter. The D code of a RIN is assigned a value between 3 and 7 under current regulations. The value assigned depends on the fuel type, feedstock and production process requirements as set out in Table 1 to 40 C.F.R. §80.1426. Examples of fuels assigned a D code of 3-7 under current regulations are provided below. These examples are for illustration purposes only and are not to be considered limiting to the invention.

TABLE 2
D code examples

| D code | Fuel Type | Example |
|---|---|---|
| 3 | Cellulosic biofuel | Ethanol from cellulosic biomass from |
| | | agricultural residues |
| 4 | Biomass-based diesel | Biodiesel and renewable diesel from soy |
| | | bean oil |
| 5 | Advanced biofuel | Ethanol from sugarcane |
| 6 | Renewable fuel | Ethanol from corn starch |
| | (conventional biofuel) | |
| 7 | Cellulosic diesel | Diesel from cellulosic biomass from |
| | | agricultural residues |

As set out previously, the RFS2 mandate volumes are set by four separate but nested category groups, namely renewable biofuel, advanced biofuel, cellulosic biofuel and biomass-based diesel. The requirements for each of the nested category groups are provided in Table 3.

The nested category groups are differentiated by the D code of a RIN. To qualify as a total advanced biofuel, the D code assigned to the fuel is 3, 4, 5 or 7, while to qualify as cellulosic biofuel the D code assigned to the fuel is 3 or 7 (Table 3).

According to current regulations, each of the four nested category groups requires a performance threshold in terms of GHG reduction for the fuel type. In order to qualify as a renewable biofuel, a fuel is required to meet a 20% life cycle GHG emission reduction (or be exempt from this requirement), while advanced biofuel and biomass-based diesel are required to meet a 50% life cycle GHG emission reduction and cellulosic biofuels are required meet a 60% life cycle GHG emission reduction, relative to a gasoline baseline. As well, each nested category group is subject to meeting certain feedstock criteria. As set out previously, the advanced biofuel nested category group excludes ethanol made from corn starch, which is only a renewable fuel.

TABLE 3
Nested category groups under RFS2

| | | Life cycle GHG |
|---|---|---|
| Nested | | threshold reduction |
| category | | relative to |
| group | Fuel type | gasoline baseline |
| Renewable | Conventional biofuels (D code 6) | 20% |
| biofuel | and advanced biofuels (D code 3, | |
| | 4, 5 or 7) | |
| Advanced | Cellulosic biofuels (D code 3 or | 50% |
| biofuel | 7), biomass-based diesel (D code | |
| | 4 or 7), and other advanced | |
| | biofuels (D code 5) | |
| Cellulosic | Biofuel derived from | 60% |
| biofuels | lignocellulosic material (D code 3) | |
| | and bio-diesel derived | |
| | lignocellulosic material (D code 7). | |
| Biomass-based | Conventional biodiesel (D code 4) | 50% |
| diesel | or cellulosic diesel (D code 7) | |

Advantageously, by displacing geologic carbon dioxide with biogenic carbon dioxide in one or more sites that use carbon dioxide in an industrial application, in accordance with the invention, and by using a feedstock that is not starch from corn, a fermentation fuel producer can produce a fuel or fuel intermediate having lower life cycle GHG emissions and in some embodiments can generate an advanced biofuel RIN associated with the fuel or fuel intermediate produced in their facility than could otherwise be generated. For example, a corn ethanol fuel producer that produces ethanol that only qualifies for a RIN having a D code of 6 can generate a RIN having a D code of 5 by switching to a non-corn starch feedstock, such as wheat or sorghum, and by using the biogenic carbon dioxide evolved during the ethanol fermentation to displace geologic carbon dioxide in an industrial application. Such a fuel can meet the feedstock criteria and the 50% GHG emission reduction threshold to qualify for an advanced biofuel, which in turn allows for the generation of a RIN having a D code of 5. Qualification of a fuel for a RIN having a D code of 5 is particularly advantageous as such RINs generally possess higher market value than those having a D code of 6, and thus can yield higher prices when traded with another party and/or sold to an obligated party. It should be appreciated that, further GHG reduction measures, in addition to those provided by the invention, can be employed to meet the threshold GHG reductions to qualify for a desired RIN or more LCFS credits. Such measures may include, without limitation, reducing plant consumption by process changes or substituting energy sources at the plant to lower GHG intensive sources such as biogas or renewable electricity.

Thus, according to certain embodiments of the invention, a RIN credit containing information or a value corresponding to a reduction in life cycle GHG emissions relative to a baseline is generated with the production of a volume of the fermentation based fuel. The information may correspond to a reduction in life cycle GHG emissions of at least 40, 45, 50, 55, 60, 65, 70, 75, 80 or 85% relative to a gasoline baseline. As set out above, the invention may contribute wholly or in part to achieving reductions in the life cycle GHG emissions of the fuel or fuel intermediate relative to a gasoline baseline.

The RIN associated with the fermentation based fuel or fuel intermediate may be assigned a D code of 3, 4, 5 or 7, also referred to herein as a D3, D4, D5 and D7 RIN, respectively. According to certain embodiments, the RIN associated with the fermentation based fuel or fuel intermediate may be assigned a D code of 3 or 5. Under current regulations, this corresponds to cellulosic biofuel and advanced biofuel fuel types, which meet GHG emissions reductions of 60% and 50%, respectively, relative to a gasoline baseline. This excludes ethanol from corn starch, which under current regulations is assigned a D code of 6. Preferably, the RIN associated with the fermentation based fuel is assigned a D code of 5.

According to a further embodiment of the invention, the fermentation based fuel may qualify for a D code having a lower numerical value than could otherwise be achieved by not practicing the invention. For example, a fuel, including but not limited to fuel made from wheat or sorghum, may be assigned a D code of 5 instead of 6 by carrying out the displacement in accordance with the invention.

In alternative embodiments of the invention, corn starch may be used as a feedstock to produce the fermentation based fuel, with the proviso that the fermentation based fuel is not ethanol. According to such embodiments, other alcohols, such as butanol or isobutanol, may be produced from corn starch.

According to some embodiments of the invention, a RIN is characterized as containing numerical information or data associated with the fuel or fuel intermediate produced by the process of the invention. Thus, a party may generate RINs comprising numerical information relating to an amount of fuel or fuel intermediate representing at least three parameters selected from (i) the type of renewable fuel that the fuel or fuel intermediate is; (ii) the year in which the fuel or fuel intermediate was produced or the year the numerical information was produced; (iii) registration number associated with the producer or importer of the fuel or fuel intermediate; and (iv) serial number associated with a batch of the fuel or fuel intermediate. The numerical information may also include one or more of the following parameters selected from: (i′) a number identifying that the numerical information is assigned to a volume of fuel or fuel intermediate, or separated; (ii′) a registration number associated with the facility at which the fuel or fuel intermediate was produced or imported; (iii′) a number representing a value related to an equivalence value of the fuel or fuel intermediate; (iv′) a number representing a first-volume numerical information associated with a batch of the fuel or fuel intermediate; and (v′) a number representing a last-volume numerical information associated with a batch of the fuel or fuel intermediate.

The RIN or numerical information described herein or portion thereof is provided to a government regulatory agency, including the EPA, in connection with generating a RIN. In some embodiments of the invention, the numerical information is also provided to a purchaser of the fermentation based fuel or a fuel derived therefrom. The numerical information described herein or portions thereof may be stored electronically in computer readable format.

The purchaser of the fermentation based fuel, or a fuel derived therefrom, may separate the RIN. As set out above, separation of a RIN from a volume of the alcohol, or a fuel derived therefrom, means termination of the assignment of the RIN to a volume of fuel. RIN separation is typically carried out by a fuel blender, importer or other obligated party. According to current regulations, when a RIN is separated, the K code of the RIN is changed to 2.

Separation of RINs may be conducted in accordance with prevailing rules and regulations, as currently provided in 40 C.F.R. §80.1129 and 40 C.F.R. §80.1429. RINs generated in accordance with the invention may be separated and subsequently traded.

It should be understood that the regulations under EISA, including RIN requirements and the criteria for categorization of a fuel under a particular fuel category, such as life cycle GHG emission thresholds, are described herein in accordance with current regulations and that the invention is not limited to current rules and will provide benefits in relation to subsequent rule changes thereof.

### Low Carbon Fuel Standard

The beneficial GHG emissions reductions achieved by the present invention also can provide a methodology for meeting low carbon fuel standards established by jurisdictions within the United States or other government authorities. The credit, which includes a certificate, may be associated with the fermentation based fuel, or a fuel derived therefrom, and represents or is proportional to the amount of life cycle GHG emissions reduced measured relative to a gasoline baseline. As set forth previously, the life cycle GHG emissions under low carbon fuel standards are often referred to as carbon intensity or CI. Preferably, the credit is associated with the improved production process for making an alcohol.

California's LCFS currently requires that all mixes of fuel that oil refineries and distributors sell in the Californian market meet in aggregate the established targets for GHG emissions reductions. California's LCFS requires increasing annual reductions in the average life cycle emissions of most transportation fuels, up to a reduction of at least 10% in the carbon intensity, which is a measure of the life cycle GHG emissions, by 2020. Targets can be met by trading of credits generated from the use of fuels with a lower GHG emission value than a gasoline baseline. Similar legislation has been implemented by the province of British Columbia, Canada, the United Kingdom and by the European Union.

British Columbia approved a Renewable and Low Carbon Fuel Requirements Act, which requires parties who manufacture or import the fuel into the province ensure that the renewable content and the average carbon intensity of the fuel they supply meets levels set by regulations. Fuel suppliers are required to submit annual reports regarding the renewable fuel content and carbon intensity of the transportation fuels they supply. The province allows transfers of GHG credits between fuel suppliers to provide flexibility in meeting the requirements of the regulation (http://www2.gov.bc.ca/).

In the European Union, GHG emissions are regulated by a Fuel Quality Directive, 98/70/EC. In April 2009, Directive 2009/30/EC was adopted which revises the Fuel Quality Directive 98/70/EC. The revisions include a new element of legislation under Article 7a that requires fuel suppliers to reduce the GHG intensity of energy supplied for road transport (Low Carbon Fuel Standard). In particular, Article 7a specifies that this reduction should amount to at least 6% by 31 Dec. 2020, compared to the EU-average level of life cycle GHG emissions per unit of energy from fossil fuels in 2010. According to the Fuel Quality Directive, fuel/energy suppliers designated by member states of the European Union are required to report to designated authorities on: (a) the total volume of each type of fuel/energy supplied, indicating where the fuel/energy was purchased and its origin; and (b) the life cycle GHG emissions per unit of energy. The European Union has also promoted the use of biofuels through a Biofuel Directive (2003/30/EC), which mandates countries across the EU to displace certain percentages of transportation fuel with biofuels by target dates.

The United Kingdom has a Renewable Transport Fuel Obligation in which biofuel suppliers are required to report on the level of carbon savings and sustainability of the biofuels they supplied in order to receive Renewable Transport Fuel Certificates (RTFCs). Suppliers report on both the net GHG savings and the sustainability of the biofuels they supply according to the appropriate sustainability standards of the feedstocks from which they are produced and any potential indirect impacts of biofuel production, such as indirect land-use change or changes to food and other commodity prices that are beyond the control of individual suppliers. Suppliers that do not submit a report will not be eligible for RTFCs.

Certificates or credits can be claimed when renewable fuels are supplied and fuel duty is paid on them. At the end of the obligation period, these certificates may be redeemed to the RTFO Administrator to demonstrate compliance.

The present invention will be further illustrated in the following examples. However, it is to be understood that the examples below are for illustrative purposes only and should not be construed to limit the current invention in any manner. Further, it should be appreciated that the values used in the GHG life cycle calculations in the examples below may be updated over time by regulatory bodies. Accordingly, the standards for determining GHG life cycle values presented herein and calculations made thereunder are exemplary and merely reflect GHG accounting modeling methods used currently by regulators.

### EXAMPLES

### Example 1

### Reducing the Life Cycle GHG Emissions Associated with a Liquid Fuel by Collecting Biogenic Carbon Dioxide and Displacing Geologic Carbon Dioxide in an Industrial Application

This example illustrates how a dry mill ethanol plant processing sorghum to ethanol can reduce its life cycle GHG emissions to below 50% of the gasoline baseline value used by the EPA under EISA by collecting biogenic carbon dioxide and displacing geologic carbon dioxide in or associated with a site that uses carbon dioxide in an industrial application. Advantageously, by meeting this GHG emission threshold, the ethanol qualifies for D5 RINs under the RFS.

In this example, the life cycle GHG emissions of the fuels are compared using EPA GHG emissions methods and their 2022 scenario for certain GHG values (see EPA-HQ-OAR-2011-0542; FRL-9680-8, Notice of Data Availability Concerning Renewable Fuels Produced From Grain Sorghum Under the RFS Program). The percentage GHG reductions relative to the gasoline baseline are calculated based on a midpoint of a range of results in accordance with Federal Register, Vol. 77, No. 113, Proposed Rules (Jun. 12, 2012), “Notice of Data Availability Concerning Renewable Fuels Produced From Grain Sorghum Under the RFS Program”, page 34923, http://www.gpo.gov/fdsys/pkg/FR-2012-06-12/pdf/2012-13651.pdf, accessed Jun. 12, 2012.

(a) Life Cycle GHG Emissions Reductions without Biogenic Carbon Dioxide Collection and Displacement of Geologic Carbon Dioxide

The following illustrates the GHG emissions associated with ethanol production from sorghum in which biogenic carbon dioxide is released to the atmosphere, also referred to as a production process baseline. As shown below, when biogenic carbon dioxide is released to the atmosphere rather than collected and used to displace geologic carbon dioxide in an industrial application, the GHG emissions of the fuel are only reduced by 32% relative to the gasoline baseline. The gasoline baseline is a 2005 gasoline baseline value as set out in EPA-HQ-OAR-2011-0542; FRL-9680-8, supra.

In the life cycle of the fuel, carbon dioxide from the atmosphere is sequestered into the starch by the action of photosynthesis. However, energy is used and GHG emissions occur during the course of the feedstock production and harvesting, the transport to the ethanol plant, the production process itself, the transport of the products to market, and the combustion of the fuel. There is also a GHG emissions increase associated with implied indirect land use changes and other indirect impacts associated with the feedstock markets. The direct carbon dioxide emissions from the fermentation of the starch and from the combustion of the ethanol are offset by carbon dioxide sequestered in the starch by photosynthesis.

Provided below is a summary of the GHG emissions that result from the ethanol production process itself. The ethanol plant uses natural gas and non-renewable electricity, and the use of these energy sources leads to the life cycle GHG emissions in Table 4.

TABLE 4
GHG emissions from the ethanol production process

| | | Value for | Emissions from |
|---|---|---|---|
| | BTU/gal | emissions, g | fuel use, g |
| | ethanol | CO₂eq/MMBTU | CO₂eq/MMBTU |
| | produced | fuel used | ethanol produced |
| Natural gas use | 17,341 | 68,575 | 15,647 |
| Biogas use | 0 | 364 | 0 |
| Non-renewable | 2,235 | 219,824 | 6,465 |
| electricity use | | | |
| TOTAL | 19,576 | | 22,111 |

The life cycle GHG emissions for ethanol production from sorghum throughout the fuel life cycle are shown below in Table 5 and compared to those of a 2005 gasoline baseline (see EPA-HQ-OAR-2011-0542; FRL-9680-8, supra). The life cycle emissions are for grain sorghum ethanol produced in plants that use natural gas and produce an industry average of 92% wet distillers grain.

TABLE 5
Life cycle GHG emissions for the gasoline baseline
and the production process baseline using grain sorghum
as the feedstock (g CO₂eq/MMBTU)

| | 2005 | Grain sorghum |
|---|---|---|
| | gasoline | ethanol (production |
| Fuel Process | baseline | process baseline) |
| Net agriculture | | 12,698 |
| Land use change | | 27,620 |
| Fuel production | 19,200 | 22,111 |
| Fuel and feedstock transport | * | 3,661 |
| Tailpipe emissions | 79,004 | 880 |
| Total emissions | 98,204 | 66,971 |
| Percent savings vs. gasoline | | 32% |

* Emissions included in fuel production stage

As can be seen in Table 5, when the emission values from each stage of the fuel life cycles are summed, the net carbon dioxide emissions values are 98,204 g CO₂ eq/MMBTU for gasoline and 66,971 g CO₂ eq/MMBTU for ethanol produced from sorghum without any biogenic carbon dioxide collection and displacement of geologic carbon dioxide with the collected biogenic carbon dioxide in an industrial application. This represents a GHG emissions reduction of only 32% relative to the gasoline baseline for ethanol produced from sorghum. Thus, the emissions fall short of the requirement to achieve D5 RINs or 50% GHG savings relative to the gasoline baseline.

(b) Life Cycle GHG Emissions Reductions with Biogenic Carbon Dioxide Collection and Displacement of Geologic Carbon Dioxide

The following illustrates that the decrease in emissions associated with the use of the invention permits an ethanol plant to achieve a 56% savings in life cycle GHG emissions associated with ethanol production relative to the gasoline baseline. This is a significant improvement from the 32% life cycle GHG savings for ethanol production without the displacement of the invention. In this example, the total quantity of biogenic carbon dioxide produced from the ethanol fermentation using sorghum as the feedstock is 2,899 g carbon dioxide per gallon of ethanol, or 38,145 g carbon dioxide per MMBTU of ethanol produced. The ethanol plant then collects 80 wt % of the biogenic carbon dioxide and uses it to displace geologic carbon dioxide in an industrial application. In this example, the biogenic carbon dioxide is transported to the industrial site using a fungible carbon dioxide pipeline. It should be appreciated that, in terms of the beneficial environmental attributes associated with the use of carbon dioxide, it is immaterial whether the displacement of geologic carbon dioxide occurs in a fungible carbon dioxide pipeline, or in a specific industrial site that draws carbon dioxide from that pipeline. Thus, certain carbon dioxide withdrawn from such a pipeline will still be considered to possess the GHG emission attributes set out below.

The ethanol plant uses natural gas and renewable electricity in the production process (per the baseline), and additional electricity and diesel fuel for the production and transport of the carbon dioxide by truck to the site that uses carbon dioxide in an industrial application. The additional usage of electricity is assumed to be 163 kWhr/ton of CO₂ collected, and the usage of diesel is based on a 390 mile one-way distance, 5 miles per gallon diesel usage, and 17.2 ton CO₂/truck. The assumed emission factor for electricity is 219,824 g CO₂ eq/MMBTU, and the assumed factor for diesel is 97,006 g CO₂ eq/MMBTU. The total quantity of biogenic carbon dioxide collected is 30,516 g carbon dioxide per MMBTU of ethanol produced (80% of 38,145).

The carbon dioxide losses associated with collection, purification, compression and transport are also accounted for and a summary of the net energy inputs to each of these operations are as follows:

TABLE 6
Carbon dioxide emissions from purification, compression
and transport of biogenic carbon dioxide

| | | | Emissions from |
|---|---|---|---|
| | Usage | Value for | fuel use, g |
| | BTU/ton | emissions, g | CO₂eq/MMBTU |
| | of CO₂ | CO₂eq/MMBTU | ethanol produced |
| Non-renewable | 556,156 | 219,824 | 3,731 |
| electricity use | | | |
| Diesel for transport | 1,174,426 | 97,006 | 3,477 |
| TOTAL | 1,730,582 | | 7,207 |

The value for the total net reduction in emissions due to displacement of geologic carbon dioxide for biogenic carbon dioxide, taking into account the collection, purification, compression and transport, is 23,308 g CO₂ eq/MMBTU ethanol. The value is arrived at by subtracting the 7,207 g CO₂ eq/MMBTU emission due to these losses, from the net GHG saving of 30,516 g CO₂ eq/MMBTU of ethanol emission due to displacement.

The net carbon dioxide emissions and savings throughout the full fuel life cycle implementing the invention are shown in Table 7 below (rightmost column). The values for the GHG savings are shown in brackets (negative emission). The net carbon dioxide emission value for the full fuel life cycle with displacement of biogenic carbon dioxide for geologic carbon dioxide is 43,662 g CO₂ eq/MMBTU ethanol, while the carbon dioxide emission value for the production process baseline is 66,971 g CO₂ eq/MMBTU ethanol. This represents a percent reduction verses the gasoline baseline of 56%, which is a significant increase relative to the 32% reduction when there is no such displacement.

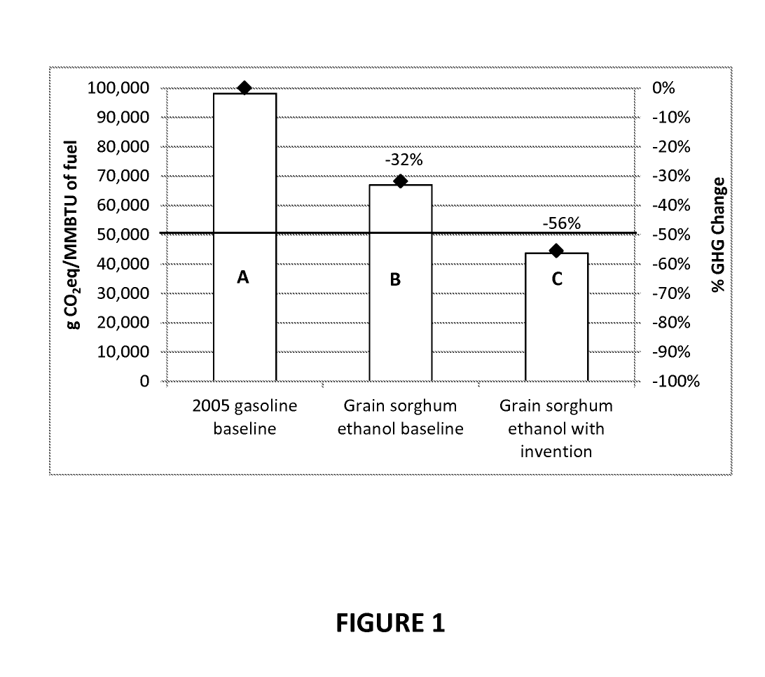

The percent changes in life cycle emissions with and without implementation of the invention are depicted in FIG. 1.

TABLE 7
Comparison of life cycle GHG emissions for gasoline baseline, production
process baseline, emissions due to the displacement of the invention
and full life cycle emissions of the fuel with the displacement

| | | | Grain sorghum ethanol |
|---|---|---|---|
| | | | with displacement of |
| | | | geologic carbon dioxide |
| | | Grain sorghum ethanol | with biogenic carbon |
| | 2005 | baseline (production | dioxide in accordance |
| | gasoline | process baseline; g | with the invention (g |
| Fuel Process | baseline | CO₂eq/MMBTU) | CO₂eq/MMBTU) |
| Net agriculture | — | 12,698 | 12,698 |
| Land use change | — | 27,620 | 27,620 |
| Fuel production | 19,200 | 22,111 | 22,111 |
| Fuel and feedstock | * | 3,661 | 3,661 |
| transport | | | |
| Tailpipe emissions | 79,004 | 880 | 880 |
| Net change from | — | — | (23,308) |
| implementation of | | | |
| the invention | | | |
| Total emissions | 98,204 | 66,971 | 43,662 |
| Midpoint life cycle | — | 32% | 56% |
| GHG reduction | | | |
| percent compared | | | |
| to gasoline | | | |

* Emissions included in fuel production stage

### Example 2

### Using the Invention to Increase the Generation of LCFS Credits in a Biogas Derived Fuel

The present invention also allows a landfill gas collection operation using biomethane from landfill organic waste to make compressed natural gas (CNG) for vehicle use, and achieve a greater degree of LCFS credit generation from the operation, as shown below. The calculations are based on California's LCFS regulations.

### (a) Baseline Emissions for Natural Gas Based CNG

The California Air Resources Board (CARB) has determined life cycle GHG emissions values for CNG derived from natural gas and CNG from landfill biomethane as in Table 8 below.

TABLE 8
Life cycle GHG emissions values for CNG
derived from natural gas and biomethane

| | | LCFS credits |
|---|---|---|
| | Emissions | generated |
| | value (g | by fuel use |
| Fuel | CO₂eq/MJ) | (g CO₂eq/MJ) |
| California Gasoline | 95.86 | 0 |
| CNG derived from natural gas | 67.7 | 28.16 |
| CNG derived from landfill biomethane | 11.3 | 84.56 |

### (b) Emission Reductions Due to the Invention

Anaerobic digestion of organic material in a landfill operation produces biomethane and carbon dioxide, although other gases such as hydrogen and impurities may be generated as well. The total quantity of carbon dioxide produced from the fermentation of organic material in the landfill operation is 49.4 g CO₂ eq per MJ of methane produced.

According to this example, the landfill operation implements the invention by collecting 80 wt % of the carbon dioxide evolved during anaerobic digestion and using the carbon dioxide to displace geologic carbon dioxide in an industrial application. The landfill operation uses renewable electricity in the production process, and diesel fuel for the transport of the carbon dioxide by truck to the site that uses carbon dioxide in the industrial application. The total quantity of biogenic carbon dioxide collected is 39.5 g carbon dioxide per MJ of biogas produced (80% of 49.4 g CO₂ eq/MJ) and the displacement of geologic carbon dioxide by biogenic carbon dioxide leads to a reduction of carbon dioxide emissions of 39.5 g CO₂ eq per MJ of biogas produced.

The energy used and the GHG emissions that occur as a result of the carbon dioxide collection, compression and transport are also accounted for. The GHG impact of these operations leads to an increase of 8.56 g CO₂ eq per MJ of biogas. A summary of the net energy inputs to and emissions associated with the collection, compression, and transport operations are as follows:

TABLE 9
Carbon dioxide emissions from purification, compression
and transport of biogenic carbon dioxide

| | | | Emissions from |
|---|---|---|---|
| | MJ | CARB value for | fuel use, g |
| | energy/MJ | emissions, g | CO2eq/MJ |
| | biogas | CO₂eq/MJ | biogas produced |
| Renewable electricity | 0.023 | 0 | 0 |
| use | | | |
| Diesel for transport | 0.090 | 94.71 | 8.56 |
| TOTAL | 0.114 | | 8.56 |

The net life cycle GHG savings associated with the implementation of the invention is 30.94 g CO₂ eq/MJ biogas (savings of 39.50 g CO₂ eq/MJ offset by an increase of 8.56 g CO₂ eq/MJ).

(c) Combined Emissions from Fuel Life Cycle

The decrease in emissions associated with the use of the invention in this example permits the landfill to generate additional LCFS credits equal to 30.94 g CO₂ eq/MJ of CNG-based biogas, when compared to CNG based biogas without use of the invention. The LCFS credit value of the CNG based biogas is increased from 84.56 g CO₂ eq/MJ to 115 g CO₂ eq/MJ, an increase of 36.6% by use of the invention. Table 10 below summarizes the baselines and the changes in g CO₂ eq/MJ.

TABLE 10
Comparison of the emissions values and LCFS credits for California
gasoline, CNG derived from natural gas and CNG derived from landfill
biogas with displacement of biogenic carbon dioxide

| | | LCFS credits |
|---|---|---|
| | Emissions | generated |
| | value (g | by fuel use |
| Fuel | CO₂eq/MJ) | (g CO₂eq/MJ) |
| California gasoline | 95.86 | 0 |
| CNG derived from natural gas | 67.7 | 28.16 |
| CNG derived from landfill biomethane | 11.3 | 84.56 |
| Incremental impact of CO₂ sequestration | (39.5) | 39.50 |
| Incremental impact of CO₂ processing and | 8.56 | (8.56) |
| transport | | |
| CNG derived from landfill biomethane | (19.64) | 115.50 |
| with invention | | |

### Example 3

### Reducing the Life Cycle GHG Emissions Associated with a Liquid Fuel by Using Methane Having Reduced Life Cycle GHG Emissions

The present invention also enables a liquid fuel production facility, such as an ethanol production facility, to reduce the life cycle GHG emissions of the liquid fuel by using methane having reduced life cycle GHG emissions to provide energy to the production facility or associated utilities.

According to this example, biomethane and biogenic carbon dioxide is generated in a landfill by anaerobic digestion and the biomethane is then separated from the carbon dioxide. The carbon dioxide that is collected is used in an industrial application to displace geologic carbon dioxide, while the biomethane is supplied for use in the liquid fuel production facility or utilities to generate energy in the form of heat or electricity. The decrease in emissions associated with the use of such low GHG methane for energy production permits the liquid fuel production facility to generate a liquid fuel having a D5 RIN. In this example, the liquid fuel is ethanol produced from sorghum.

### (a) Introducing the Biomethane to a Pipeline and Withdrawing Methane Having Reduced GHG Emissions at the Liquid Fuel Production Facility

In this example, the biomethane is introduced to a natural gas pipeline that supplies methane to the ethanol fuel production facility.

Since the pipeline is fed by natural gas, as well as biomethane, the methane withdrawn may not contain actual molecules from the original organic material from which the biomethane is derived, but rather the energy equivalent value of the biomethane. With respect to biomethane used for electricity generation in a facility, government authorities have recognized that it is immaterial, in terms of the beneficial environmental attributes associated with the use of biomethane, whether the displacement of fossil fuel occurs in a fungible natural gas pipeline, or in a specific fuel production facility that draws methane from that pipeline. Thus, methane withdrawn from such a pipeline will still be considered to possess the GHG emission reductions set out in Example 3(c) below.

The amount of biomethane fed to the natural gas pipeline and the amount of methane withdrawn from such a pipeline is the same and is determined by gas metering. A gas meter is placed at the point on the pipeline where biomethane is introduced and another meter is placed at the point on the pipeline where methane is withdrawn for use in the fuel production facility. A contract is in place which sets out the amount of biomethane fed to the pipeline by the landfill operation and the amount of methane to be withdrawn for use at the ethanol production facility.

### (b) Using Methane Having Reduced GHG Emissions to Supply Energy to the Ethanol Production Facility

The following compares the life cycle GHG emissions associated with ethanol production from sorghum using methane derived from the following processes:

(i) methane derived from biogas in which the carbon dioxide that is separated from the biogas is released to the atmosphere; and
(ii) methane derived from biogas in which the carbon dioxide that is separated from the biogas is supplied to a site that uses carbon dioxide in an industrial application to displace geologic carbon dioxide, as set out in Example 3(a) above.

Provided below in Table 11 and Table 12 is a summary of the GHG emissions that result from the ethanol production process using the methane derived from biogas from each of the above sources.

As can be seen in Table 12 below, when the emission values from each stage of the fuel life cycle are summed, the net carbon dioxide emissions value for ethanol production using methane derived from biogas in which carbon dioxide is released to the atmosphere is 51,407 g CO₂ eq/MMBTU. This represents a GHG emission reduction of only 48% relative to the gasoline baseline. This value is not sufficient for the ethanol produced in the fuel production facility to qualify for a D5 RIN. (As discussed previously, such a RIN requires a 50% reduction relative to the gasoline baseline).

TABLE 11
GHG emissions from the ethanol production process using
methane derived from biogas without CO₂ collection

| | | Value for | Emissions from |
|---|---|---|---|
| | BTU/gal | emissions, g | fuel use, g |
| | ethanol | CO₂eq/MMBTU | CO₂eq/MMBTU |
| | produced | fuel used | ethanol produced |
| Natural gas use | — | — | — |
| Biogas use | 17,341 | 364 | 83 |
| Non-renewable | 2,235 | 219,824 | 6,465 |
| electricity use | | | |
| TOTAL | 19,576 | — | 6,548 |

TABLE 12
Life cycle GHG emissions for grain sorghum ethanol process
using methane derived from biogas without CO₂ collection

| | 2005 | Grain sorghum | Grain sorghum |
|---|---|---|---|
| | gasoline | ethanol | ethanol |
| Fuel Process | baseline | baseline | using biogas |
| Net agriculture | — | 12,698 | 12,698 |
| Land Use Change | — | 27,620 | 27,620 |
| Fuel Production | 19,200 | 22,111 | 6,548 |
| Fuel and Feedstock | * | 3,661 | 3,661 |
| Transport | | | |
| Tailpipe emissions | 79,004 | 880 | 880 |
| Total emissions | 98,204 | 66,971 | 51,407 |
| Midpoint life cycle | — | 32% | 48% |
| GHG reduction | | | |
| percent compared | | | |
| to gasoline | | | |

* Emissions included in fuel production stage

### (c) Production of Methane Having Reduced Life Cycle GHG Emissions

The life cycle GHG emissions of the methane used in an ethanol fuel production process are reduced relative to a biomethane production process baseline by displacing geologic carbon dioxide with biogenic carbon dioxide collected from biogas as described in Table 13. The assumptions around the usage and emission intensity of both diesel and electricity are the same as outlined in Example 1. The biomethane production process baseline refers to the life cycle GHG emissions associated with a biogas production process conducted under identical conditions except the biogenic carbon dioxide that is separated from the biomethane is released to the atmosphere.

TABLE 13
GHG Emissions from the process of purification, compression
and transport of biogenic carbon dioxide

| | | | Emissions from |
|---|---|---|---|
| | Usage | Value for | fuel use, g |
| | BTU/ton | emissions, g | CO₂eq/MMBTU |
| | CO₂ | CO₂eq/MMBTU | ethanol produced |
| Non-renewable | 556,156 | 219,824 | 1,162 |
| electricity use | | | |
| Diesel for transport | 1,1744,26 | 97,006 | 1,083 |
| TOTAL | 293,927 | | 2,245 |

As stated in Example 2, 80 wt % of the carbon dioxide produced during anaerobic digestion is collected. The total quantity of biogenic carbon dioxide collected is 41,667 g carbon dioxide per MMBTU of biogas produced (80% of the 52,084 CO₂ eq/MMBTU which is produced in the landfill operation). This equates to a value of 9,507 g CO₂ eq per MMBTU of ethanol produced. The GHG emissions associated with carbon dioxide collection, compression and transport account for an increase of 2,245 g CO₂ eq/MMBTU of ethanol (Table 13).

The value for the total net reduction from the invention is 7,262 g CO₂ eq/MMBTU ethanol. The value is obtained by subtracting the 2,245 g CO₂ eq/MMBTU emission due to these losses, from the net GHG saving of 9,507 g CO₂ eq/MMBTU of ethanol emission due to displacement.

Table 14 below summarizes the baselines and the changes. It is noted that when using methane in a fuel production facility that is derived from biogas in which the carbon dioxide that is separated from the biomethane is supplied to a site that uses carbon dioxide in an industrial application to displace geologic carbon dioxide, the sum of the GHG emissions is 44,144 g CO₂ eq/MMBTU. This represents a GHG emissions reduction of 55% relative to the gasoline baseline. Due to these significant life cycle GHG emission reductions relative to the gasoline baseline, the ethanol produced from the fuel production facility meets the GHG emission reduction threshold needed to qualify for a D5 RIN.

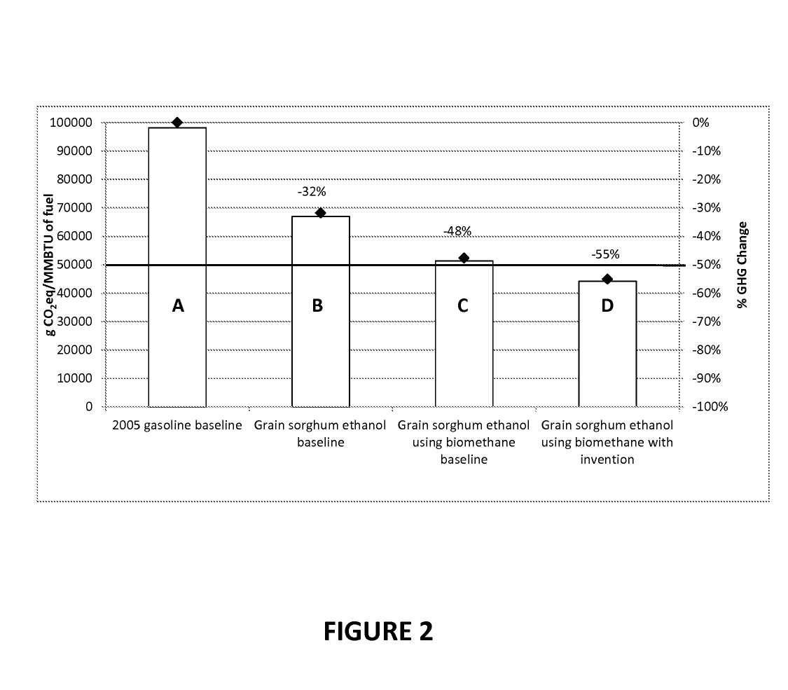

The percent changes in life cycle emissions with and without implementation of the invention are depicted in FIG. 2.

TABLE 14
Comparison of the emissions values for the gasoline baseline,
methane derived from natural gas and methane derived from landfill
biogas with displacement of biogenic carbon dioxide

| | | | Grain sorghum | Grain sorghum ethanol |
|---|---|---|---|---|
| | | | ethanol using | using biomethane from |
| | 2005 | | biomethane | which biogenic carbon |
| | gasoline | Grain sorghum | production process | dioxide is collected |
| Fuel Process | baseline | ethanol baseline | baseline | and used to displace |
| Net agriculture | — | 12,698 | 12,698 | 12,698 |
| Land use change | — | 27,620 | 27,620 | 27,620 |
| Fuel production | 19,200 | 22,111 | 6,548 | 6,548 |
| Fuel and feedstock | * | 3,661 | 3,661 | 3,661 |
| transport | | | | |
| Tailpipe emissions | 79,004 | 880 | 880 | 880 |
| Change from | — | — | — | (7,262) |
| implementation | | | | |
| of the invention | | | | |
| Total emissions | 98,204 | 66,971 | 51,407 | 44,144 |
| Midpoint lifecycle | — | 32% | 48% | 55% |
| GHG reduction | | | | |
| percent compared | | | | |
| to gasoline | | | | |

* Emissions included in fuel production stage

1. A process for reducing life cycle GHG emissions associated with production of a liquid fuel or fuel intermediate comprising:
(i) producing sugar from plant derived organic material;
(ii) fermenting the sugar to produce biogenic carbon dioxide and the liquid fuel or fuel intermediate;
(iii) collecting an amount of biogenic carbon dioxide generated from the step of fermenting;
(iv) supplying the biogenic carbon dioxide from step (iii) for use in one or more sites that use carbon dioxide in an industrial application, and causing displacement of geologic carbon dioxide;
(v) recovering the liquid fuel or fuel intermediate produced by the step of fermenting;
(vi) generating a renewable fuel credit associated with the liquid fuel or fuel intermediate; and
(vii) prior to step (vi), generating or receiving data representative of a life cycle GHG emission reduction of the liquid fuel or fuel intermediate relative to a gasoline baseline,
wherein the life cycle GHG emissions associated with the production of the liquid fuel or fuel intermediate are reduced by at least 1.5 g CO₂ eq/MJ relative to a production process baseline as a result of the displacement.

2. The process of claim 1, wherein the one or more sites use carbon dioxide as an additive, a processing agent, a treatment agent, a cooling agent, or a carbon source to make fuels, chemicals or polymers.

3. The process of claim 1, wherein the one or more sites use carbon dioxide as an additive to a food, a beverage or water; a processing agent to process a food or food ingredient; a carbon source to make a carbonate or methanol; or as a cooling agent in food processing or preservation.

4. The process of claim 1, wherein the displacement results from taking out of use a first amount of geologic carbon dioxide at the one or more sites that use carbon dioxide in an industrial application and subsequently using the biogenic carbon dioxide supplied in step (iv) to displace the first amount of geologic carbon dioxide.

5. The process of claim 1, wherein the liquid fuel or fuel intermediate is ethanol derived from wheat or sorghum.

6. The process of claim 1, wherein the renewable fuel credit generated in step (vi) is a renewable identification number.

7. The process of claim 6, wherein the renewable identification number has a D code value of 3 or 5.

8. The process of claim 1, wherein the renewable fuel credit generated in step (vi) is a low carbon fuel credit.

9. The process of claim 1, wherein the displacement within step (iv) results from:
(a) introducing the biogenic carbon dioxide into an apparatus for transporting carbon dioxide to one or more sites that used or are using geologic carbon dioxide in an industrial application; or
(b) supplying the biogenic carbon dioxide for use in one or more sites that used or are using geologic carbon dioxide in an industrial application.

10. The process of claim 1, wherein the step of supplying comprises introducing the biogenic carbon dioxide into apparatus for transporting said biogenic carbon dioxide to the one or more sites, wherein in respect of at least one or more of the sites at least two conditions are met selected from:
(a) the site has used geologic carbon dioxide in the industrial application;
(b) the site has access to geologic carbon dioxide for use in the industrial application; and
(c) written documentation indicates that biogenic carbon dioxide is used to displace geologic carbon dioxide.

11. The process of claim 10, wherein in respect of at least one or more of the sites, written documentation indicates that biogenic carbon dioxide is used to displace geologic carbon dioxide.

12. The process of claim 1, wherein the data representative of a life cycle GHG emission reduction of the liquid fuel or fuel intermediate relative to a gasoline baseline is determined by a quantification of a GHG emission reduction due to a reduction in the use of geologic carbon dioxide in the one or more sites that occurred or would occur over the lifetime of the one or more sites as a result of the use of biogenic carbon dioxide.

13. A process for generating a D5 RIN credit associated with ethanol produced in an ethanol production facility, said process comprising using a non-corn starch feedstock to supply the production facility and carrying out the process of claim 1 to reduce the life cycle GHG emissions of the ethanol to a level relative to a gasoline baseline sufficient to qualify for the D5 RIN credit.

14. The process of claim 1, wherein the supplying comprises introducing an amount of biogenic carbon dioxide into an apparatus for delivering carbon dioxide to one or more sites that use carbon dioxide in an industrial application and causing a third party to withdraw from said apparatus an amount of carbon dioxide less than or at least approximately equal to the amount of carbon dioxide introduced to said apparatus.

15. The process of claim 1, the supplying comprises introducing an amount of biogenic carbon dioxide into an apparatus for delivering carbon dioxide to one or more sites that use carbon dioxide in an industrial application and causing a third party to withdraw from said apparatus an amount of carbon dioxide, wherein the withdrawn carbon dioxide has GHG emission attributes associated therewith that are the same as the GHG emission attributes of the biogenic carbon dioxide introduced to the apparatus.

16. The process of claim 1, wherein step (vii) is carried out by a third party.

17. A process comprising:
(i) receiving carbon dioxide for use at a site that uses carbon dioxide in an industrial application, said carbon dioxide produced by the process of claim 1; and
(ii) using the carbon dioxide received in step (i) to displace geologic carbon dioxide.

18. The process of claim 17, wherein the carbon dioxide received for use at the site is produced by a third party.

19. The process of claim 17, wherein receiving the carbon dioxide comprises withdrawing an amount of carbon dioxide from an apparatus for delivering carbon dioxide to one or more sites that use carbon dioxide in an industrial application, said apparatus having had introduced thereto an amount of the biogenic carbon dioxide, wherein the carbon dioxide withdrawn has GHG emission attributes associated therewith that are the same as the GHG emission attributes of the biogenic carbon dioxide introduced to the apparatus.

20. The process of claim 17, wherein receiving the carbon dioxide comprises withdrawing an amount of carbon dioxide from an apparatus for delivering carbon dioxide to one or more sites that use carbon dioxide in an industrial application, said apparatus having had introduced thereto an amount of the biogenic carbon dioxide, wherein the amount of carbon dioxide withdrawn is an amount less than or at least approximately equal to the amount of carbon dioxide introduced to said apparatus.

21. A process comprising:
(a) withdrawing an amount of carbon dioxide from an apparatus for delivering carbon dioxide to one or more sites that use carbon dioxide in an industrial application, said apparatus having had introduced thereto an amount of biogenic carbon dioxide derived from a fermentation that produces a liquid fuel or fuel intermediate using organic material as a feedstock;
which carbon dioxide withdrawn has GHG emission attributes associated therewith that are the same as the GHG emission attributes of the biogenic carbon dioxide introduced to the apparatus; and
(b) using the carbon dioxide withdrawn in step (a) to displace geologic carbon dioxide.

22. The process of claim 21, wherein the amount of carbon dioxide withdrawn is less than or at least approximately equal to the amount of biogenic carbon dioxide introduced to the apparatus.

23. The process of claim 21, wherein a third party introduces the biogenic carbon dioxide into the apparatus.

24. The process of claim 21, further comprising causing the amount of biogenic carbon dioxide to be introduced to said apparatus for delivering carbon dioxide to one or more sites that use carbon dioxide in an industrial application.

25. The process of claim 21, wherein the GHG emission attributes of the withdrawn carbon dioxide are set out in written documentation.

26. The process of claim 25, wherein the written documentation comprises data describing a life cycle GHG analysis indicating that the displacement of geologic carbon dioxide creates a net GHG benefit.

27. The method of claim 21, wherein the displacement of step (b) results from taking out of use a first amount of geologic carbon dioxide at the site that uses carbon dioxide in an industrial application and subsequently using the carbon dioxide that is withdrawn to displace the first amount of geologic carbon dioxide.

28. The process of claim 21, wherein the biogenic carbon dioxide is sourced from a fuel production facility that generates renewable fuel credits associated with producing a liquid fuel.

29. A process to reduce the life cycle GHG emissions associated with production of a liquid fuel or fuel intermediate, said process comprising:
(i) producing sugar from plant derived organic material and converting the sugar to the liquid fuel or fuel intermediate in a fuel production facility;
(ii) using methane to supply energy in any part of the fuel production facility or associated utilities, wherein said methane has associated with it life cycle GHG emissions that are reduced relative to a biomethane production process baseline as a result of the practice of:
(a) anaerobically digesting plant derived organic material to produce biogas comprising biomethane and biogenic carbon dioxide;
(b) separating the biomethane and biogenic carbon dioxide;
(c) collecting an amount of the biogenic carbon dioxide generated from the step of separating;
(d) supplying the biogenic carbon dioxide from step (c) to one or more sites that use carbon dioxide in an industrial application, and causing displacement of geologic carbon dioxide; and
(e) supplying the biomethane to an apparatus for delivering methane to one or more fuel production facilities;
(iii) recovering the liquid fuel or fuel intermediate;
(iv) generating a renewable fuel credit associated with the liquid fuel or fuel intermediate; and
(v) prior to step (iv), generating or receiving data representative of a life cycle GHG emission reduction of the liquid fuel or fuel intermediate relative to a gasoline baseline,
wherein the life cycle GHG emissions associated with the production of the biomethane are reduced by at least 5 g CO₂ eq/MJ relative to a biomethane production process baseline as a result of the displacement of the geologic carbon dioxide.

30. The process of claim 29, wherein steps (a)-(e), step (v) or steps (a)-(e) and step (v) are practiced by one or more third parties.

31. The process of claim 29, wherein the one or more sites use carbon dioxide as an additive, a processing agent, a treatment agent, a cooling agent, or a carbon source to make fuels, chemicals or polymers.

32. The process of claim 29, wherein the one or more sites use carbon dioxide as an additive to a food, a beverage or water; a processing agent to process a food or food ingredient; a carbon source to make a carbonate or methanol; or as a cooling agent in food processing or preservation.

33. The process of claim 29, wherein the displacement within step (d) results from taking out of use a first amount of geologic carbon dioxide at the one or more sites that use carbon dioxide in an industrial application and subsequently using the biogenic carbon dioxide supplied in step (d) to displace the first amount of geologic carbon dioxide.

34. The process of claim 29, wherein the liquid fuel or fuel intermediate is an alcohol.

35. The process of claim 29, wherein the liquid fuel or fuel intermediate is ethanol derived from sorghum or wheat.

36. The process of claim 29, wherein the renewable fuel credit generated in step (iv) is a renewable identification number.

37. The process of claim 36, wherein the renewable identification number has a D code value of 3 or 5.

38. The process of claim 29, wherein the renewable fuel credit generated in step (iv) is a low carbon fuel credit.

39. The process of claim 29, wherein the displacement of step (d) results from:
(i′) introducing the biogenic carbon dioxide into an apparatus for transporting carbon dioxide to one or more sites that used or are using geologic carbon dioxide in an industrial application; or
(ii′) supplying the biogenic carbon dioxide for use in one or more sites that used or are using geologic carbon dioxide in an industrial application.

40. The process of claim 29, wherein the step of supplying comprises introducing the biogenic carbon dioxide into apparatus for transporting said biogenic carbon dioxide to the one or more sites, wherein in respect of at least one or more of the sites at least two conditions are met selected from:
(a′) the site has used geologic carbon dioxide in the industrial application;
(b′) the site has access to geologic carbon dioxide for use in the industrial application; and
(c′) written documentation indicates that biogenic carbon dioxide is used to displace geologic carbon dioxide.

41. The process of claim 40, wherein in respect of at least one or more of the sites, written documentation indicates that biogenic carbon dioxide is used to displace geologic carbon dioxide.

42. The process of claim 29, wherein the data representative of a life cycle GHG emission reduction of the liquid fuel or fuel intermediate relative to a gasoline baseline is determined by a quantification of a GHG emission reduction due to a reduction in the use of geologic carbon dioxide in the one or more sites that occurred or would occur over the lifetime of the one or more sites as a result of the use of biogenic carbon dioxide.

43. The process of claim 29, wherein the methane used to supply energy in any part of the fuel production facility or associated utilities is withdrawn from a natural gas pipeline containing methane from sources other than anaerobic digestion of organic material.

44. The process of claim 29, wherein the methane supplies energy in the form of heat or electricity.

45. The process of claim 29, wherein the sugar is converted to the fuel or fuel intermediate by a fermentation that produces biogenic carbon dioxide and wherein the process further comprises collecting an amount of biogenic carbon dioxide produced from the step of fermenting and supplying the biogenic carbon dioxide that is collected for displacement of geological carbon dioxide.

46. A process for generating a D5 RIN credit associated with ethanol produced in an ethanol production facility, said process comprising using a non-corn starch feedstock to supply the ethanol production facility and carrying out the process of claim 29 to reduce the life cycle GHG emissions of the ethanol to a level relative to a gasoline baseline sufficient to qualify for the D5 RIN credit.

47. The process of claim 29, wherein the supplying comprises introducing an amount of biogenic carbon dioxide into an apparatus for delivering carbon dioxide to one or more sites that use carbon dioxide in an industrial application and causing a third party to withdraw from said apparatus an amount of carbon dioxide and wherein the withdrawn carbon dioxide has GHG emission attributes associated therewith that are the same as the GHG emission attributes of the biogenic carbon dioxide introduced to the apparatus.

48. The process of claim 29, wherein the supplying comprises introducing an amount of biogenic carbon dioxide into an apparatus for delivering carbon dioxide to one or more sites that use carbon dioxide in an industrial application and causing a third party to withdraw from said apparatus an amount of carbon dioxide and wherein the amount of carbon dioxide withdrawn is less than or at least approximately equal to the amount of carbon dioxide introduced to said apparatus.

49. A process comprising:
(i) receiving carbon dioxide at a site that uses carbon dioxide in an industrial application, said carbon dioxide produced by the process of claim 29; and
(ii) using the carbon dioxide received in step (i) to displace geologic carbon dioxide.

50. The process of claim 49, wherein a third party produces the carbon dioxide that is received at the site.

51. The process of claim 49, wherein receiving the carbon dioxide comprises withdrawing an amount of carbon dioxide from an apparatus for delivering carbon dioxide to one or more sites that use carbon dioxide in an industrial application, said apparatus having had introduced thereto an amount of the biogenic carbon dioxide and wherein the carbon dioxide withdrawn has GHG emission attributes associated therewith that are the same as the GHG emission attributes of the biogenic carbon dioxide introduced to the apparatus.

52. The process of claim 49, wherein receiving the carbon dioxide comprises withdrawing an amount of carbon dioxide from an apparatus for delivering carbon dioxide to one or more sites that use carbon dioxide in an industrial application, said apparatus having had introduced thereto an amount of the biogenic carbon dioxide and wherein the amount of carbon dioxide withdrawn is an amount less than or at least approximately equal to the amount of carbon dioxide introduced to said apparatus.

53. A process comprising:
(a) withdrawing an amount of carbon dioxide from an apparatus for delivering carbon dioxide to one or more sites that use carbon dioxide in an industrial application, said apparatus having had introduced thereto an amount of biogenic carbon dioxide derived from an anaerobic digestion of organic material,
the carbon dioxide withdrawn having GHG emission attributes associated therewith that are the same as the GHG emission attributes of the biogenic carbon dioxide introduced to the apparatus; and
(b) using the carbon dioxide withdrawn in step (a) at a site that uses carbon dioxide in the industrial application to displace geologic carbon dioxide.

54. The process of claim 53, wherein the amount of carbon dioxide withdrawn is less than or at least approximately equal to the amount of biogenic carbon dioxide introduced to the apparatus.

55. The process of claim 53, further comprising causing the amount of biogenic carbon dioxide to be introduced to said apparatus for delivering carbon dioxide to one or more sites that use carbon dioxide in an industrial application.

56. The process of claim 53, wherein the GHG emission attributes of the withdrawn carbon dioxide are set out in written documentation.

57. The process of claim 53, wherein the written documentation comprises data describing a life cycle GHG analysis indicating that the displacement of geologic carbon dioxide creates a net GHG benefit.

58. The process of claim 53, wherein the displacement in step (b) results from taking out of use a first amount of geologic carbon dioxide at the site that uses carbon dioxide in an industrial application and subsequently using the carbon dioxide that is withdrawn to displace the first amount of geologic carbon dioxide.

59. A process comprising:
(i) receiving carbon dioxide for use at a site that uses carbon dioxide in an industrial application, said carbon dioxide supplied from an anaerobic digestion that produces biogas comprising biomethane and biogenic carbon dioxide; and
(ii) using the carbon dioxide received in step (i) to displace geologic carbon dioxide.

60. The process of claim 59, wherein receiving the carbon dioxide comprises withdrawing an amount of carbon dioxide from a pipeline for delivering carbon dioxide to one or more sites that use carbon dioxide in an industrial application and wherein the carbon dioxide withdrawn has GHG emission attributes associated therewith that are the same as the GHG emission attributes of the biogenic carbon dioxide introduced to the apparatus.

61. The process of claim 59, wherein receiving the carbon dioxide comprises withdrawing an amount of carbon dioxide from a pipeline for delivering carbon dioxide to one or more sites that use carbon dioxide in an industrial application, said pipeline having had introduced thereto an amount of the biogenic carbon dioxide and wherein the amount of carbon dioxide withdrawn is an amount less than or at least approximately equal to the amount of carbon dioxide introduced to said pipeline.

62. A process comprising:
(i) providing an amount of biogenic carbon dioxide generated from a fermentation process to produce a liquid fuel or fuel intermediate;
(ii) supplying the biogenic carbon dioxide from step (i) to one or more sites that use carbon dioxide in an industrial application;
(iii) generating data or receiving data in written documentation from a third party, said data being representative of a life cycle GHG emission reduction of the liquid fuel or fuel intermediate resulting from the fermentation relative to a gasoline baseline, wherein
said data demonstrates a reduction in emissions due to displacement of geologic carbon dioxide, and
said data is stored in digital format in a storage medium used to retain digital data, and
the life cycle GHG emissions associated with the production of the liquid fuel or fuel intermediate are reduced by at least 1.5 g CO₂ eq/MJ relative to a production process baseline as a result of the displacement,
(iv) recovering the liquid fuel or fuel intermediate produced by the fermentation process; and
(v) generating a renewable fuel credit associated with the liquid fuel or fuel intermediate.

63. A process for reducing life cycle GHG emissions associated with production of a liquid fuel or fuel intermediate comprising:
(i) producing sugar from plant derived organic material;
(ii) fermenting the sugar to produce biogenic carbon dioxide and the liquid fuel or fuel intermediate;
(iii) collecting an amount of biogenic carbon dioxide generated from the step of fermenting;
(iv) supplying the biogenic carbon dioxide from step (iii) to one or more sites that use carbon dioxide in an industrial application, and causing displacement of geologic carbon dioxide;
wherein the life cycle GHG emissions associated with the production of the liquid fuel or fuel intermediate are reduced by at least 1.5 g CO₂ eq/MJ relative to a production process baseline as a result of the displacement;
(v) recovering the liquid fuel or fuel intermediate produced by the step of fermenting;
(vi) generating or receiving data relating to a quantity of carbon dioxide displaced or a life cycle GHG emission analysis of the liquid fuel or fuel intermediate resulting from the fermentation; and
(vii) generating a renewable fuel credit associated with the liquid fuel or fuel intermediate.

64. The process of claim 63, wherein the supplying comprises introducing an amount of biogenic carbon dioxide into an apparatus for delivering carbon dioxide to one or more sites that use carbon dioxide in an industrial application and causing a third party to withdraw from said apparatus an amount of carbon dioxide and wherein the carbon dioxide withdrawn has GHG emission attributes associated therewith that are the same as the GHG emission attributes of the biogenic carbon dioxide introduced to the apparatus.

65. The process of claim 63, wherein the supplying comprises introducing an amount of biogenic carbon dioxide into an apparatus for delivering carbon dioxide to one or more sites that use carbon dioxide in an industrial application and causing a third party to withdraw from said apparatus an amount of carbon dioxide less than or at least approximately equal to the amount of carbon dioxide introduced to said apparatus.

66. A process to reduce the life cycle GHG emissions associated with production of a liquid fuel or fuel intermediate, said process comprising:
(i) producing sugar from plant derived organic material and converting the sugar to the liquid fuel or fuel intermediate in a fuel production facility;
(ii) using methane to supply energy in any part of the fuel production facility or associated utilities, wherein said methane has associated with it life cycle GHG emissions that are reduced relative to a biomethane production process baseline as a result of the practice of:
(a) anaerobically digesting plant derived organic material to produce biogas comprising biomethane and biogenic carbon dioxide;
(b) separating the biomethane and biogenic carbon dioxide;
(c) collecting an amount of the biogenic carbon dioxide generated from the step of separating;
(d) supplying the biogenic carbon dioxide from step (c) to one or more sites that use carbon dioxide in an industrial application, and causing displacement of geologic carbon dioxide; and
(e) supplying the biomethane to an apparatus for delivering methane to one or more fuel production facilities;
wherein the life cycle GHG emissions associated with the production of the biomethane are reduced by at least 5 g CO₂ eq/MJ relative to a biomethane production process baseline as a result of the displacement of geologic carbon dioxide;
(iii) recovering the liquid fuel or fuel intermediate;
(iv) generating data or receiving data relating to a quantity of carbon dioxide displaced or a life cycle GHG emission analysis of the liquid fuel or fuel intermediate; and
(v) generating a renewable fuel credit associated with the liquid fuel or fuel intermediate.

67. The process of claim 66, wherein a third party practices steps (a)-(e).

68. The process of claim 66, wherein the supplying in step (d) comprises introducing an amount of biogenic carbon dioxide into an apparatus for delivering carbon dioxide to one or more sites that use carbon dioxide in an industrial application and causing a third party to withdraw from said apparatus an amount of carbon dioxide and wherein the carbon dioxide withdrawn has GHG emission attributes associated therewith that are the same as the GHG emission attributes of the biogenic carbon dioxide introduced to the apparatus.

69. The process of claim 66, wherein the supplying in step (d) comprises introducing an amount of biogenic carbon dioxide into an apparatus for delivering carbon dioxide to one or more sites that use carbon dioxide in an industrial application and causing a third party to withdraw from said apparatus an amount of carbon dioxide less than or at least approximately equal to the amount of carbon dioxide introduced to said apparatus.
